(19) United States
(12) Patent Application Publication
(10) Pub. No.: US 2026/0009799 A1
(43) Pub. Date: Jan. 8, 2026

(54) CA-IX TARGETING FLUORESCENT PROBES

(71) Applicant: BRACCO IMAGING S.P.A., Milan (IT)

(72) Inventors: Roberta NAPOLITANO, Albiano d'Ivrea (IT); Francesco BLASI, Colleretto Giacosa (IT); Federica CHIANALE, Chivasso (IT); Erika REITANO, Torino (IT); Alessia ADAMO, Banchette (TO) (IT)

(73) Assignee: BRACCO IMAGING S.P.A., Milan (IT)

(21) Appl. No.: 18/872,210

(22) PCT Filed: Jul. 26, 2023
(86) PCT No.: PCT/EP2023/070685

§ 371 Date: Dec. 5, 2024

(30) Foreign Application Priority Data
Jul. 28, 2022 (EP) 22187427.4

(51) Int. Cl. G01N 33/58; A61K 49/00; C09B 23/08; G01N 33/573

(52) U.S. Cl. CPC G01N 33/582; A61K 49/0032; A61K 49/0052; C09B 23/086; G01N 33/573; G01N 2333/765; G01N 2333/988

(57) ABSTRACT

The present invention relates to the field of optical imaging. More particularly, it relates to fluorescent probes targeting carbonic anhydrase nine (CA-IX) comprising near-infrared (NIR) dyes of the cyanine. The invention also relates to the methods for preparing these compounds, to pharmaceutical compositions and kits incorporating them and to methods of use them as optical diagnostic agents in imaging or therapy of diseases such as solid tumors with hypoxic tissues involving cells expressing CA-IX.

### FIELD OF THE INVENTION

The present invention relates to the field of optical imaging. More particularly, it relates to fluorescent probes able to efficiently target Carbonic Anhydrase IX enzyme (CA-IX) and comprising heptamethine cyanine dyes with near-infrared (NIR) emission. The invention also relates to methods for preparing these compounds, to pharmaceutical compositions and kits incorporating them and to methods of use them as optical diagnostic agents in imaging or therapy of diseases, particularly solid tumors with hypoxic tissues involving cells expressing CA-IX.

### BACKGROUND ART

Dyes are chemical entities that absorb photons of a specific wavelength upon light excitation and re-emit some of that energy, depending on quantum efficiency, usually at a longer wavelength. Particularly, cyanine dyes are fluorescent organic molecules characterized by a delocalized electron system that spans over a polymethine bridge and is confined between two nitrogen atoms. Given the favorable optical properties, low toxicity, and good solubility in aqueous media, cyanine dyes can be used as contrast agents for biomedical imaging. Cyanine dyes emitting in the Near-Infra Red (NIR) region (700-900 nm) are particularly useful for biomedical imaging applications due to the high penetration depth compared to dyes with fluorescence emission in the visible spectrum.

Among the NIR dyes currently used for biomedical imaging, Indocyanine green (ICG) is a medicinal product approved for human use that may accumulate in pathological regions thanks to different homing mechanisms. Fluorophores like Fluorescein and ICG distribute in tumor tissues by a combination of passive diffusion and enhanced permeability and retention (EPR) effect (Onda N. et al., Int J Cancer 2016, 139, 673-682). However, this first generation of fluorescent contrast agents requires a large mass dose (>1 mg/kg) for proper visualization. Moreover, false positive and false negative responses are common clinical findings associated with the use of these contrast agents (Tummers Q. et al., PlosOne 2015).

A second generation of contrast agents for optical imaging is currently under development, exploiting the use of a dye conjugated to a targeting agent to improve sensitivity and specificity of detection. Molecular vectors such as small molecules, peptides, mAbs, antibody fragments, confer tumor selective properties to the probes, whereas the fluorescent dyes work as detection moieties for spatial localization (Gioux S. et al., Mol Imaging 2010, 9(5), 237-255). However, the in vivo behavior of dye-biomolecule conjugates may be strongly affected by the biological properties of the fluorescent moiety. For example, small structural modifications of a cyanine Cy5 strongly modulate the accumulation in tumor and off-target tissues of the relative bioconjugates (Bunschoten A. et al., Bioconjugate Chem. 2016, 27, 1253-1258). Fluorescent contrast agents with low non-specific accumulation and high selectivity for the target tissue would be preferable for applications in living organisms.

Several examples of known cyanine dyes conjugated with low molecular weight targeting moieties are disclosed in the background art. For instance, one of the targets of utmost interest for its potential for clinical settings is Carbonic Anhydrase IX (CA-IX).

CA-IX is a homo-dimeric glycoprotein located on the cell surface (Hilvo et al., J Biol Chem 2008; 283(41): 27799-27809) and is a member of the large carbonic anhydrases (CAs) family of zinc metalloenzymes. These enzymes are involved in the maintenance of cellular acid-base homeostasis through the catalysis of the reversible hydration of carbonic dioxide (CO₂+H₂O), which is converted to protons and bicarbonate (HCO³⁻+H⁺), leading to a local decrease in pH.

To date, sixteen CA isoforms have been characterized in mammals, which differ in their cellular localization, catalytic activity, susceptibility to different inhibitors and tissue-specific distribution.

Among them, CA-IX has been demonstrated to be an excellent biomarker of the hypoxic response in tumors, because its gene expression is promoted by the master regulator of hypoxia, named Hypoxia Inducible Factor 1 (HIF-1), believed to be involved in maintaining the acidic environment of hypoxic cells (Wykoff et al., Cancer Res. 2000, 60, 7075-7083). Tumor hypoxia, mostly resulting from poor perfusion and anemia, is one of the key factors in inducing the development of cell clones with an aggressive and treatment-resistant phenotype that leads to rapid progression and poor prognosis in several cancer types. Cancer cells, in fact, survive in a hostile environment changing their gene expression, and among them especially genes involved in pH control.

Indeed, CA-IX plays an important role in the growth and metastasis of numerous tumors (including renal cancer, cervical cancer, colon cancer, prostate cancer, breast cancer, head and neck tumors, etc.), because its catalytic activity contributes to the reduction of the extracellular pH, producing an acid microenvironment which increases cancer cell proliferation and invasion.

Unlike other CAs, many studies have demonstrated that CA-IX is expressed only in few normal tissues (namely the intestinal and stomach mucosa, gallbladder and testis), whereas it becomes overexpressed in many types of cancer cells, especially in solid tumors, characterized by insufficient oxygen supply. CA-IX thus represents an established biomarker of tumor hypoxia which plays a pivotal role in carcinogenesis and it is considered as a hallmark of the disease (Muz et al., Hypoxia (Auckl) 2015; 3: 83-92).

CA-IX targeting using specific tools could open new important fields to improve the conventional therapies and the early diagnosis and prognosis of malignant tumors.

The classes of CA-IX-targeting agents developed so far, for both imaging and/or therapeutic applications, include monoclonal antibodies (e.g. G250, M75) or mini-antibodies (e.g. A3 and CC7) and small chemical compounds, such as inorganic ions, sulfonamide-based compounds, phenols and coumarins. Some agents belonging to these classes of compounds are currently under clinical development.

In particular, the monoclonal antibodies (mab) M75 and G250 represent the first solutions developed to target CA-IX enzyme. Mab M75 binds to CA-IX's PG-like domain on the N-terminus of the target, whereas mab G250 interacts with CA-IX's catalytic domain. A chimeric version of G250 (designated cG250), labeled with the radionuclide ¹²⁴I, was developed for the detection of clear cell Renal Cell Carcinoma (ccRCC). The use of the antibody cG250 in a method of diagnosing, predicting and/or classifying a cancer disease including quantification of CA-IX expression is for instance described in WO2014/128258.

Although, typically, monoclonal antibodies have been considered as the ligands of choice for most tumor targeting applications, it is becoming increasingly clear that they are subjected to many disadvantages. In fact, antibodies are characterized by slow and inefficient tumor penetration and long blood residence, which requires the use of long-lived radioisotopes and imaging at late time points, exposing patients to a high radiation burden. Indeed, ¹²⁴I-cG250 reaches tumor/blood ratios suitable for imaging only 2-7 days after injection into the patient. Additionally, monoclonal antibodies may be immunogenic, precluding repeated administration for routine diagnostic procedures.

These problems could be circumvented with the use of small molecules. Unlike large macromolecules, small molecules clear rapidly from circulation and thus reach tumor/blood ratios suitable for imaging at early time-points. This fact in turn allows physicians to obtain diagnostic information much more quickly than with antibody-based imaging agents.

Among small chemical compounds targeting CAs, the best investigated and most robust class of inhibitors are the sulfonamides (i.e. acetazolamide) due to their high affinity, availability and ease of chemical manipulation.

While they have been in clinical use for decades for conditions such as glaucoma, seizures, altitude sickness and as a diuretic, only recently they have also been considered as antitumor agents for their capability to inhibit CA-IX. The first compound to arrive in Phase I and II clinical trials, alone or in combination with other antitumor agents, was Indisulam, but it has been discontinued. The only CA inhibitor (CAI) currently in clinical development is SLC-0111, an ureidobenzenesulfonamide that has successfully completed a phase I study, to determine safety and tolerability in patients with advanced solid tumors (McDonald et al., Am J Clin Oncol 2020; 43(7): 484-490).

For imaging purposes, CAIs have been labeled with various positron emitting isotopes (e.g. ¹⁸F, ⁶⁴Cu, ⁶⁸Ga, ¹¹¹In) or with ^{99m}Tc in order to obtain PET/SPECT agents.

Acetazolamide is an FDA-approved drug used to treat glaucoma, epilepsy, altitude sickness, periodic paralysis, heart failure, etc. Acetazolamide is a first-generation carbonic anhydrase inhibitor, hence causing the accumulation of carbonic acid, and it is known to strongly bind the Zn ion present in the catalytic pocket thanks to its terminal sulfonamide group, deprotonated as —SONH— at physiological pH.

It is possible to hydrolyze the acetyl group of acetazolamide and replace it with different linkers, for example by acylation of the amino group, still maintaining the inhibition activity and a medium/strong binding to the enzyme, depending of course on the type of substituent.

In 2015, Wichert et al. (Nat Chem 2015; 7(3): 241-249) described the selection of novel low-nanomolar binders for CA-IX by an innovative chemical library technology for affinity maturation of ligands against a defined target. Among them, the acetazolamide derivative named “4a ligand” was identified by modification via acyl chloride and click chemistry of acetazolamide, after hydrolysis of the acetyl moiety in strong acidic aqueous media. Ligand 4a displayed high affinity for CA-IX (16.7 nM, measured by SPR) and its use as delivery vehicle for tumor targeting has been reported.

Examples of NIR fluorescent dyes conjugated with “4a ligand” are disclosed for instance in WO2020/229438 (e.g. compound 11 on page 13, with a linear heptamethine dye) and WO2021/259899 (e.g. compound 6 on page 19, with a cyclohexenyl heptamethine dye).

Acetazolamide and other sulfonamides have been also conjugated to various NIR fluorescent dyes with the aim of imaging hypoxia-induced CA-IX expression in tumor cells. Indeed, among the fluorescent dyes targeting CA-IX reported in literature functionalized with different sulfonamides, even cyclic secondary amide as saccharine, the best results in terms of binding have always been obtained by derivatization of acetazolamide. A pilot in vivo experiment illustrated the potential of NIR fluorescent CAIs and fluorescence molecular tomography (FMT) imaging for non-invasive quantification of CA-IX expression (Groves et al., Bioorg Med Chem Lett 2012; 22: 653-657).

Representative examples of NIR fluorescent probes targeting CA-IX enzyme are disclosed in WO2017/161195 (On Target Laboratories), describing compounds comprising a ligand for CA-IX, a dye and a linker formed by an amino acid, amide, ureido or polyethylene glycol derivative. In particular, among the several compounds therein disclosed, it is mentioned the compound Az-8AOA-LMNIR2, also known as HypoxyFluor-1, which is currently under development as fluorescent probe for targeted imaging of tumors having high expression of CA-IX, like optical imaging and surgery involving CA-IX positive tissues or tumors. This compound is also mentioned for instance in Mahalingam S. M. et al., Bioconjugate Chem 2018, 29, 3320-3331, where its use for fluorescence imaging of hypoxic tumors is described in detail.

In this compound, as in many other compounds disclosed in the literature, the acetazolamide targeting moiety is conjugated at the central (meso) position of the heptamethine scaffold of the dye. Typically, the attachment of the ligand is performed on a central phenyl or —O-phenyl ring which is functionalized with a carboxy, carboxamido or ester group, optionally by interposition of a linker.

This central position, however, in several cases suffers from higher lability of the central substituent that could be removed in vivo by reaction with endogenous nucleophiles (i.e., it is susceptible of in vivo nucleophilic substitution), causing the loss of the targeting unit and leading to a potential in vivo degradation and de-activation of the probe. This drawback has been partially resolved by replacing the central C—O bond with a C—C bond, much more stable in vivo, but in this case the water solubility of the dye tends to dramatically decrease due to a higher tendency of aggregation.

In general, the use of tumor-targeted fluorescence dyes to help surgeons identify otherwise undetected tumor nodules, decrease the incidence of cancer-positive margins, and facilitate localization of malignant lymph nodes has been demonstrated considerable promising for improving cancer debulking surgery. Unfortunately, the repertoire of available fluorescent dyes targeting CA-IX expressing tumors is not yet extensive and diversified to permit identification of all types of hypoxic tumoral cells, raising the need to develop additional CA-IX-specific fluorescent dyes that, taking advantage from specific targeting at the bio-molecular level, may ensure localization of all malignant lesions and precise tumor excision during cancer surgeries.

Thus, despite the efforts made so far, there is still the urgent and unmet need to develop efficient fluorescent probes, targeting CA-IX with high affinity and specificity in order to be used in diagnosis and/or therapy.

Differently from the above-mentioned probes, the compounds of the present invention are characterized by Cy7 cyanine dyes conjugated to an acetazolamide moiety to a different position of the scaffold, namely at position 1 of one of the indolenine rings, and therefore they are more stable in vivo.

Moreover, the compounds of the invention have shown a surprising improvement with respect to the affinity for CA-IX target compared to other fluorescent probes bearing a CA-IX targeting moiety in the same conjugation position. Moreover, they are endowed with a lower affinity to the plasma proteins, thus displaying improved bioavailability results during in vivo trials.

### SUMMARY OF THE INVENTION

Generally, object of the present invention is to provide CA-IX targeting fluorescent probes useful as contrast agents for optical imaging and aimed at solving the above-mentioned issues. Particularly, the present invention provides fluorescent probes with optimal properties for different molecular imaging applications, being able to interact with CA-IX expressing cells, to accumulate in pathological cells and tissues and to specifically display a fluorescent signal in correspondence to pathological tissues with high signal-to-noise ratio and improved imaging efficacy at low mass doses.

The fluorescent probes of the invention are characterized by a heptamethine cyanine dye conjugated to an acetazolamide moiety through different linkers, affording strong binding results to the target and unexpectedly displaying an optimal in vitro and in vivo behaviour, thus being particularly suitable for molecular imaging.

In detail, among the several advantages that can be achieved by means of the present compounds, the following features can be highlighted for instance: high selectivity for the target tissue and low accumulation due to non-specific interaction with other tissues, high solubility in water, low binding to plasma proteins, negligible or no observed adverse reactions after systemic administration, good chemical and optical stability in plasma after administration.
A further aspect of the invention relates to such fluorescent probes for use as diagnostic agents, in particular for use in optical imaging of a human or animal organ or tissue, for use in a method of optical imaging, wherein the imaging is a tomographic imaging of organs, monitoring of organ functions including angiography, urinary tract imaging, bile duct imaging, nerve imaging, intraoperative cancer identification, fluorescence-guided surgery, fluorescence life-time imaging, short-wave infrared imaging, fluorescence endoscopy, fluorescence laparoscopy, robotic surgery, open field surgery, laser guided surgery, or a photoacoustic or sonofluorescence method.
Moreover, the invention relates to a manufacturing process for the preparation of the provided compounds and/or pharmaceutically acceptable salts thereof, and to their use in the preparation of a diagnostic agent.
According to a further aspect, the invention relates to a pharmaceutically acceptable composition comprising at least one compound of the invention, or a pharmaceutically acceptable salt thereof, in a mixture with one or more physiologically acceptable carriers or excipients. Said compositions are useful in particular as optical imaging agents to provide useful imaging of human or animal organs or tissues.
In another aspect, the present invention refers to a method for the optical imaging of a body organ, tissue or region by use of an optical imaging technique that comprises the use of an effective dose of a compound of the invention.

### DESCRIPTION OF THE INVENTION

Accordingly, a first aspect of the invention relates to a compound of formula (I), or a pharmaceutically acceptable salt thereof,

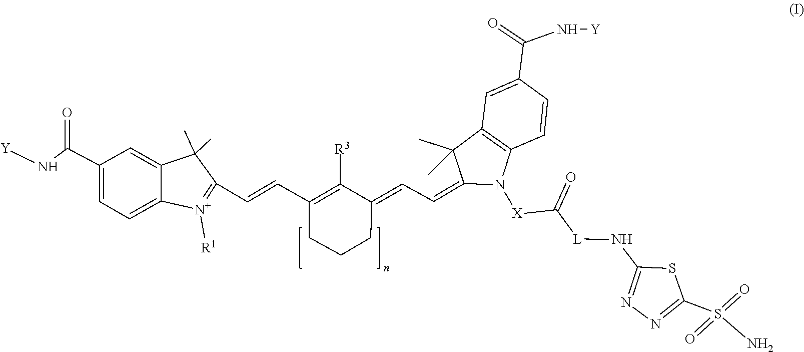

wherein
R¹ is selected from a straight or branched C₁-C₁₀ alkyl, substituted by a group —SO₃H, and a group of formula (II)

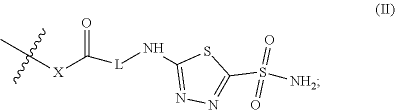

X is a straight or branched C₁-C₁₀ alkylene;
L is a bond or a linker;
each Y is independently a straight or branched C₁-C₁₀ alkyl substituted with at least two hydroxyl groups;
R³ is selected from hydrogen and a group phenyl or —O-phenyl optionally substituted with —SO₃H;
n is an integer equal to 0 or 1.

Preferably the linker L is a group of formula —NH—(CH₂)ₚ—CO— or a diradical of one or more moieties selected from the group consisting of an amino acid, such as for instance glycine, alanine, β-alanine, lysine, homolysine, ornithine, glutamic acid, aspartic acid and the like; a peptide comprising from 2 to 10 amino acids in L or D configuration; 4-aminomethylbenzoic acid; cysteic acid; a polyethylene glycol, such as a group of formula —NH—(O—CH₂—CH₂)ₚ— or —NH—(O—CH₂—CH₂)ₚ—CO— or derivatives thereof; amino-polyethylene glycol-carboxylic acid; diaminobutyric acid; and diaminopropionic acid; or it is a group -L₁-L₂- wherein L₁ is a diradical of a diamine, such as for instance a amino-polyethylene glycol amine of formula —NH—(O—CH₂—CH₂)ₚ—NH— or a diradical of ethylenediamine, propylenediamine, putrescine, spermidine, spermine, hexanediamine and the like; and L₂ is a diradical of a dicarboxylic acid, such as for instance succinic acid, glutaric acid, suberic acid, adipic acid and the like; wherein p is an integer comprised between 1 and 20.

More preferably, L is selected from a group of formula —NH—(CH₂)ₚ—CO—; a polyethylene glycol of formula —NH—(O—CH₂—CH₂)ₚ—CO—; and a diradical comprising from one to five amino acids, wherein p is an integer comprised between 1 and 20.

Preferably X is a straight or branched C₁-C₆ alkylene.

The present invention also relates to methods for preparing the compounds of formula (I) by means of synthetic transformations steps.

The invention further comprises compounds of formula (I) for use as fluorescent agents for the detection of a tumor margin in guided surgery.

### DESCRIPTION OF THE FIGURES

FIG. 1 shows representative binding curves of compound 1 and 2 of the invention, together with the ones obtained for reference compounds 1 (HF-1), 2, and 3 to CA-IX expressing HT-29 cells.

FIG. 2 shows the binding of compound 1 to HT-29 cells in competition with the fully human recombinant MSC8 antibody, selectively recognizing and blocking the CA-IX isoform. Panel A shows the binding of compound 1 (Ex 640 nm, Em 780/60 nm); panel B shows the binding of MSC8, detected through a secondary antibody (Ex 488 nm, Em 533/30 nm).

FIG. 3 shows the in vivo tumor-to-background ratio (TBR) of representative compounds 1 and 2 and of the References 1 (HF-1), 2 and 3 from images acquired 24 hours after administration.

FIG. 4 shows (a) the ex vivo tumor-to-background ratio (TBR) and (b) the organs-to-muscle ratio of representative compounds 1, 2 and of the References 1 (HF-1), 2 and 3 from samples collected 24 hours after administration.

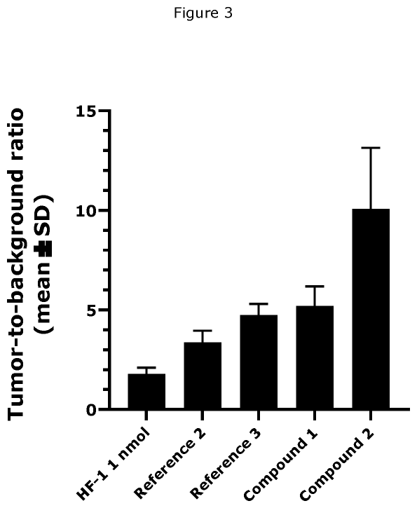

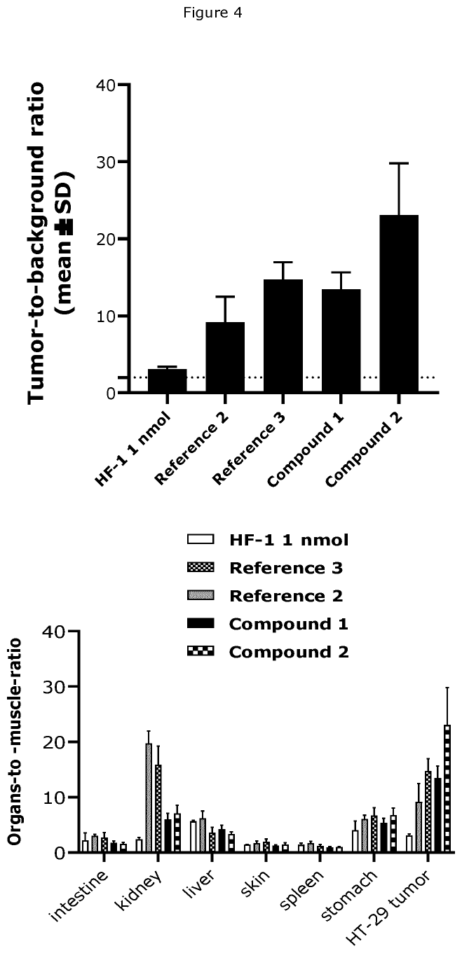

TBR statistical analysis in FIGS. 3 and 4(a) was performed using 1-way-ANOVA, Dunnett's multiple comparison test, where p<0.05 indicated significant values different from the References (1 nmol/mouse).

### Definitions

In the present description, and unless otherwise provided, the following terms and phrases as used herein are intended to have the following meanings.

The term “diradical” refers to a chemical group wherein the hydrogen atoms at two terminal portions of the molecule are removed to form a bond.

The expression “straight or branched C₁-C₁₀ alkyl” refers to an aliphatic hydrocarbon radical group, which may be a straight or branched-chain, having from 1 to 10 carbon atoms in the chain. For instance, “C₁-C₈alkyl” comprises within its meaning a linear or branched chain comprising from 1 to 8 carbon atoms. Representative and preferred alkyl groups include methyl, ethyl, n-propyl, iso-propyl, n-butyl, tert-butyl, pentyl and hexyl. Unless otherwise specified, the straight or branched alkyl is a monovalent radical group. In some cases it may be a “bivalent” or “multivalent” radical group, wherein two or more hydrogen atoms are removed from the above hydrocarbon radical group and substituted, e.g. methylene, ethylene, iso-propylene groups and the like. In such cases, the expression “straight or branched C₁-C₁₀ alkylene” is used.

The term “hydroxyalkyl” refers to any of the corresponding alkyl chain wherein one or more hydrogen atoms are replaced by hydroxyl groups.

The term “protecting group” (Pg) designates a protective group adapted for preserving the function of the group to which it is bound. Specifically, protective groups are used to preserve amino, hydroxyl or carboxyl functions. Appropriate protective groups may include, for example, benzyl, carbonyl, such as formyl, 9-fluoromethyloxycarbonyl (Fmoc), benzyloxycarbonyl (Cbz), t-butoxycarbonyl (Boc), isopropyloxycarbonyl or allyloxycarbonyl (Alloc), alkyl, e.g. tert-butyl or triphenylmethyl, sulfonyl, acetyl groups, such as trifluoroacetyl, benzyl esters, allyl, or other substituents commonly used for protection of such functions, which are well known to the person skilled in the art (see, for instance, the general reference T. W. Green and P. G. M. Wuts, Protective Groups in Organic Synthesis, Wiley, N.Y. 2007, 4^{th} Ed., Ch. 5).

Moreover, the invention comprises also the precursors or intermediates compounds suitable for the preparation of a desired compound of formula (I) or salts thereof. In such intermediates any functional group, such as a carboxylic acid or carboxamide, can be protected with an appropriate protecting group (Pg) as defined above, preferably with alkyl or ester groups. If necessary, also hydroxyl groups of Y groups can be protected with an appropriate protecting group (Pg) during the preparation of the compounds of formula (I), forming for instance acetoxy, alkoxy or ester groups.

The expression “coupling reagent” refers to a reagent used for instance in the formation of an amide bond between a carboxyl moiety and an amino moiety. The reaction may consist of two consecutive steps: activation of the carboxyl moiety and then acylation of the amino group with the activated carboxylic acid. Non limiting examples of such coupling agents are selected from the group consisting of: carbodiimides, such as N,N′-diisopropylcarbodiimide (DIC), N,N′-dicyclohexylcarbodiimide (DCC), 1-ethyl-3-(3-dimethylaminopropyl) carbodiimide (EDAC), 1-ethyl-3-(3-dimethylaminopropyl)carbodiimide (EDC) and 1-ethyl-3-(3-dimethylaminopropyl)carbodiimide (WSC); phosphonium reagents, such as (benzotriazol-1-yloxy)tris(dimethylamino)phosphonium hexafluorophosphate (BOP), (benzotriazol-1-yloxy)tripyrrolidinophosphonium hexafluorophosphate (PyBOP), 7-azabenzotriazol-1-yloxy-tripyrrolidino-phosphonium hexafluorophosphate (PyAOP), [ethyl cyano(hydroxyimino)acetato-O2]tri-1-pyrrolidinylphosphonium hexafluorophosphate (PyOxim), bromotripyrrolidinophosphonium hexafluorophosphate (PyBrOP) and 3-(diethoxyphosphoryloxy)-1,2,3-benzotriazin-4(3H)-one (DEPBT); and aminium/uronium-imonium reagents, such as N,N,N′,N′-tetramethyl-O-(benzotriazol-1-yl)uronium tetrafluoroborate (TBTU), N,N,N′,N′-tetramethyl-O-(1H-benzotriazol-1-yl)uronium hexafluorophosphate (HBTU), N,N,N′,N′-tetramethyl-O-(7-azabenzotriazol-1-yl)uronium hexafluorophosphate (HATU), O-(1H-6-chlorobenzotriazole-1-yl)-1,1,3,3-tetramethyluronium hexafluorophosphate (HCTU), 1-[1-(cyano-2-ethoxy-2-oxoethylidene-aminooxy)-dimethylamino-morpholino]-uronium hexafluorophosphate (COMU), 2-(2,5-dioxopyrrolidin-1-yl)-1,1,3,3-tetramethylisouronium tetrafluoroborate (TSTU), N,N,N′,N′-tetramethyl-O—(N-succinimidyl)uronium hexafluorophosphate (HSTU) and fluoro-N,N,N′,N′-tetramethylformamidinium hexafluorophosphate (TFFH) or other compounds well known to the person skilled in the art.

The expression “activated carboxylic acid” refers to a derivative of a carboxyl group that is more susceptible to nucleophilic attack than a free carboxyl group; suitable derivatives may include for instance acid anhydrides, thioesters, acyl halides, NHS ester and sulfo NHS esters.

The terms “moiety” or “residue” are herewith intended to define the residual portion of a given molecule once properly attached or conjugated, either directly or through a suitable linker, to the rest of the molecule.

The term “imaging agent” refers to a detectable entity that can be used in in vitro, ex vivo or in vivo visualization or detection of a biological element including cells, biological fluids and biological tissues originating from a live mammal patient, and preferably, human patient, as well as human body organ, regions or tissues, when the said detectable entity is used in association with a suitable diagnostic imaging technique.

Preferably, the fluorescent probes of the invention are able to selectively target tumor cells or tissues expressing CA-IX. In particular they are able to target tumors selected from brain cancer, breast cancer, head and neck cancer, ovarian cancer, prostate cancer, esophageal cancer, skin cancer, gastric cancer, pancreatic cancer, bladder cancer, oral cancer, lung cancer, renal cancer, uterine cancer, thyroid cancer, liver cancer, and colorectal cancer. In addition, the fluorescent probes of the invention are able to target metastatic spreads of the above-mentioned cancers in tissues and organs different from the primary source. Furthermore, the fluorescent probes of the invention are able to target pre-neoplastic lesions and dysplasia in different tissues and organs.

### Linker (L)

According to the invention, L is a linker, optionally present, that separates the CA-IX targeting ligand (acetazolamide) from the dye.

The presence of a linker is particularly useful for some embodiments where the acetazolamide moiety and the dye risk adversely interacting with each other. Moreover, the presence of the linker may be advantageous when the dye is relatively large and may interfere with the binding of the targeting moiety to the target site.

The linker can be either flexible (e.g., including linear alkyl chains) or rigid (e.g., including amino acids with aryl groups) so that the dye is oriented away from the target. The linker can also modify pharmacokinetic and metabolism of the conjugates of formula (I) used as imaging agents in a living organism.

Hydrophilic linkers may reduce the interaction with plasma proteins, reduce blood circulation time and facilitate excretion. For example, if the linker is a polyethyleneglycol (PEG) moiety, the pharmacokinetics and blood clearance rates of the imaging agent in vivo may be altered. In such embodiments, the linker can improve the clearance of the imaging agent from background tissue (i.e., muscle, blood) thus giving a better diagnostic image due to high target-to-background contrast. Moreover, the introduction of a particular hydrophilic linker may shift the elimination of the contrast agent from hepatic to renal, thus reducing overall body retention.

Therefore, in one preferred embodiment, the linker is a group of formula —NH—(CH₂)ₚ—CO— or a diradical of one or more moieties selected from the group consisting of an amino acid, such as for instance glycine, alanine, β-alanine, lysine, homolysine, ornithine, glutamic acid, aspartic acid and the like; a peptide comprising from 2 to 10 amino acids in L or D configuration; 4-aminomethylbenzoic acid; cysteic acid; a polyethylene glycol, such as a group of formula —NH—(O—CH₂—CH₂)ₚ— or —NH—(O—CH₂—CH₂)ₚ—CO— or derivatives thereof; amino-polyethylene glycol-carboxylic acid; diaminobutyric acid; and diaminopropionic acid; or it is a group -L₁-L₂- wherein L₁ is a diradical of a diamine, such as for instance a amino-polyethylene glycol amine of formula —NH—(O—CH₂—CH₂)ₚ—NH— or a diradical of ethylenediamine, propylenediamine, putrescine, spermidine, spermine, hexanediamine and the like; and L₂ is a diradical of a dicarboxylic acid, such as for instance succinic acid, glutaric acid, suberic acid, adipic acid and the like; wherein p is an integer comprised between 1 and 20.

More preferably, L is selected from a group of formula —NH—(CH₂)ₚ—CO—; a polyethylene glycol of formula —NH—(O—CH₂—CH₂)ₚ—CO—; and a diradical comprising from one to five amino acids, wherein p is an integer comprised between 1 and 20.

The compounds of the above formula (I) may have one or more asymmetric carbon atoms, otherwise referred to as chiral carbon atoms, and may thus give rise to diastereomers and optical isomers. Unless otherwise provided, the present invention further includes all such possible diastereomers as well as their racemic mixtures, their substantially pure resolved enantiomers, all possible geometric isomers, and pharmaceutically acceptable salts thereof.

The present invention further relates to compounds of the above formula (I) in which the sulfonyl group of R¹ and/or R³ may be in the form of a negatively charged ion or a pharmaceutically acceptable salt.

### DETAILED DESCRIPTION OF THE EMBODIMENTS

In a preferred embodiment the invention relates to a compound of formula (I) wherein Y is independently selected from the group consisting of

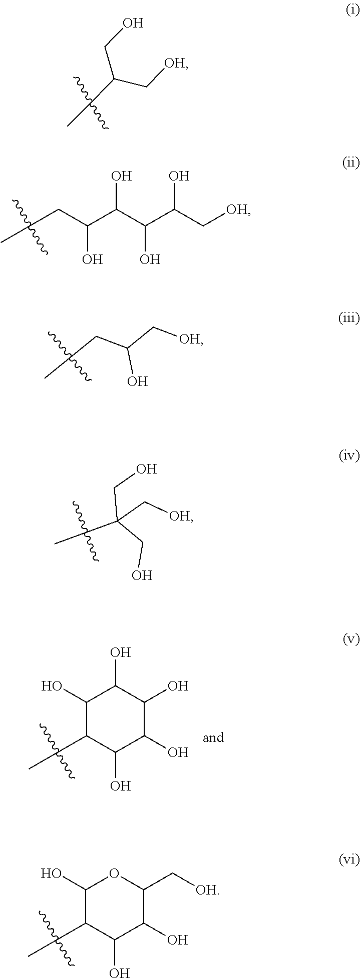 [SMILES: *C(CO)CO ; *CC(O)C(O)C(O)C(O)CO ; *CC(O)CO ; *C(CO)(CO)CO ; *C1C(O)C(O)C(O)C(O)C1O ; *C1C(O)OC(CO)C(O)C1O]

More preferably, the invention relates to a compound of formula (I) wherein Y is a group of formula (ii) as defined above. Preferably, the group (ii) has the following stereochemical configuration, obtained by using a D-glucamine in the preparation of the compounds:

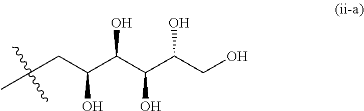 [SMILES: *C[C@H](O)[C@@H](O)[C@H](O)[C@H](O)CO]

More preferably both Y are the above group (ii).

In another preferred embodiment R¹ is a group —(CH₂)₄SO₃H or —CH₂CH₂CH(CH₃)SO₃H.

In another preferred embodiment R¹ is a group (II) as defined above.

Preferably X is a linear C₁-C₆ alkylene.

In a preferred embodiment, X is a linear C₅ alkylene and L is a group —NH(CH₂)₇—CO— according to the following formula (Ia)

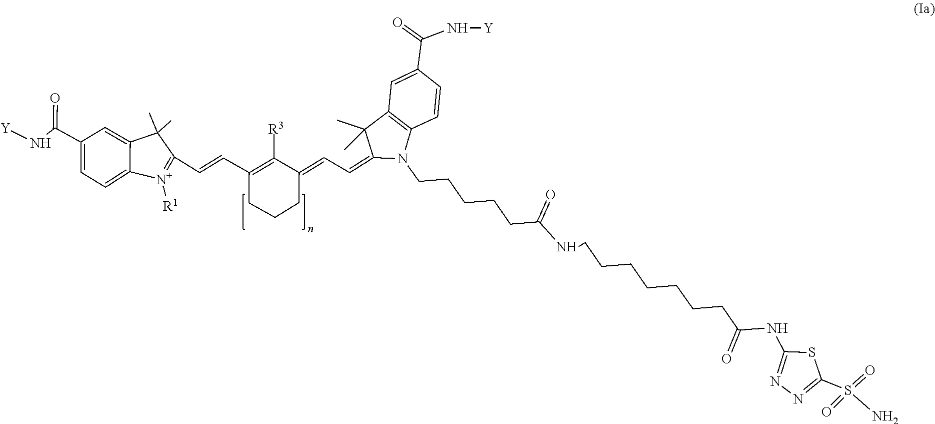

wherein R¹, R³, Y and n are as defined above.

In one preferred embodiment n is 0 and R³ is hydrogen, as represented by the following formula (Ib)

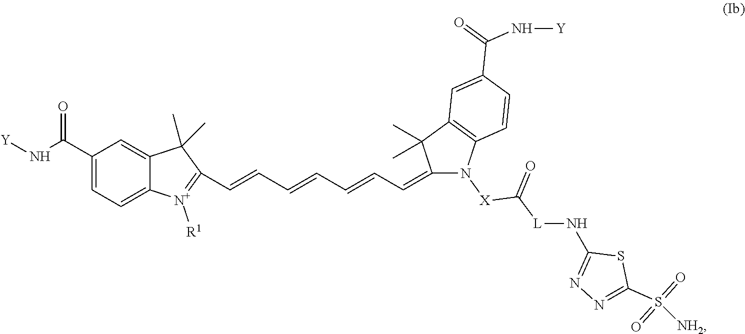

wherein R¹, X, Y and L are as defined above.

In another preferred embodiment n is 1 and R³ is a group —O-phenyl optionally substituted with —SO₃H, as represented by the following formula (Ic)

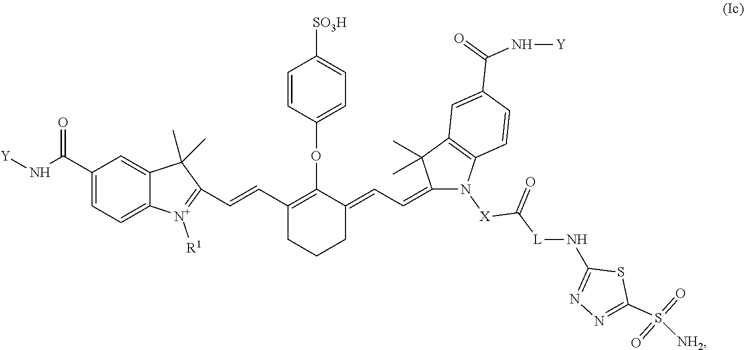

wherein R¹, X, Y and L are as defined above.

Especially preferred are the compounds of formula (I) listed in Table I.

Table I also reports the structure of the reference compound 1 corresponding to the known fluorescent probe with affinity for CA-IX Hypoxyfluor-1 (HF-1); the reference compound 2 corresponding to compound 6 disclosed in WO2021/259899; the reference compound 3 corresponding to compound 11 disclosed in WO2020/229438. Both reference compounds 2 and 3 are disclosed as CA-IX targeting probes.

TABLE I
Preferred compounds of formula (I) and reference compounds 1-3 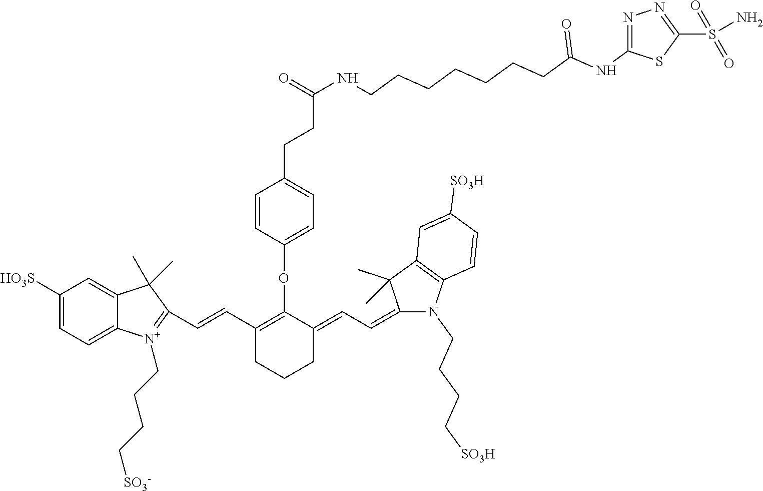
Reference 1 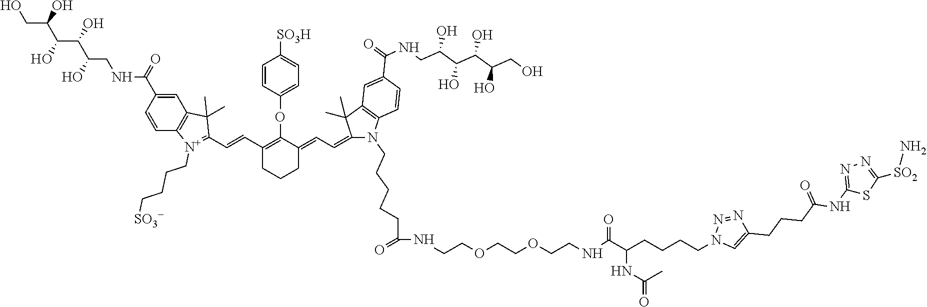
Reference 2 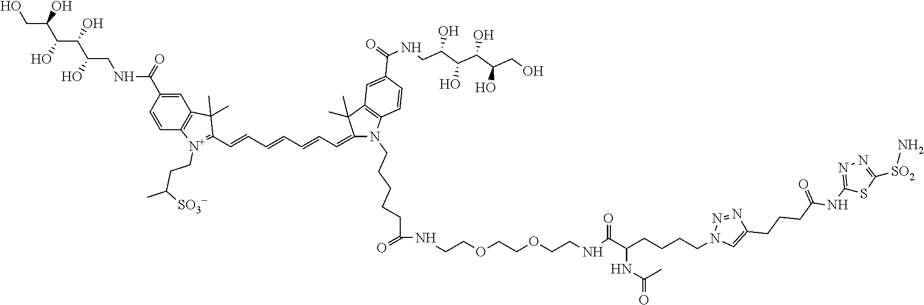
Reference 3 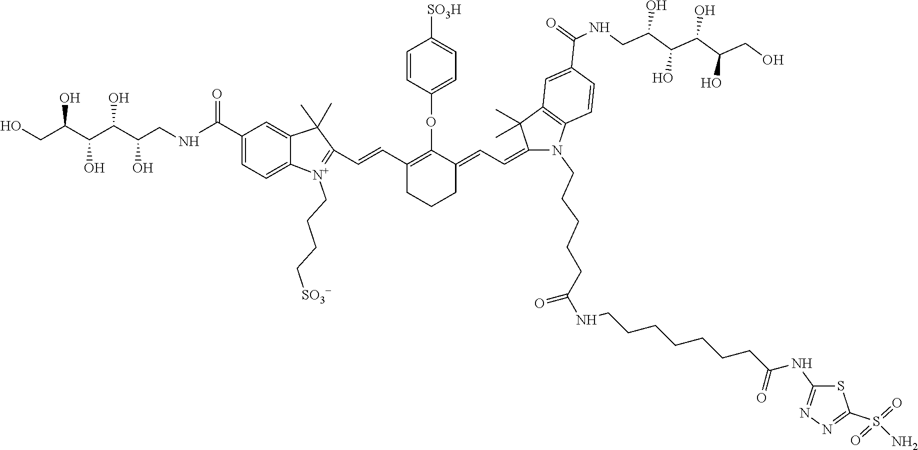
Compound 1 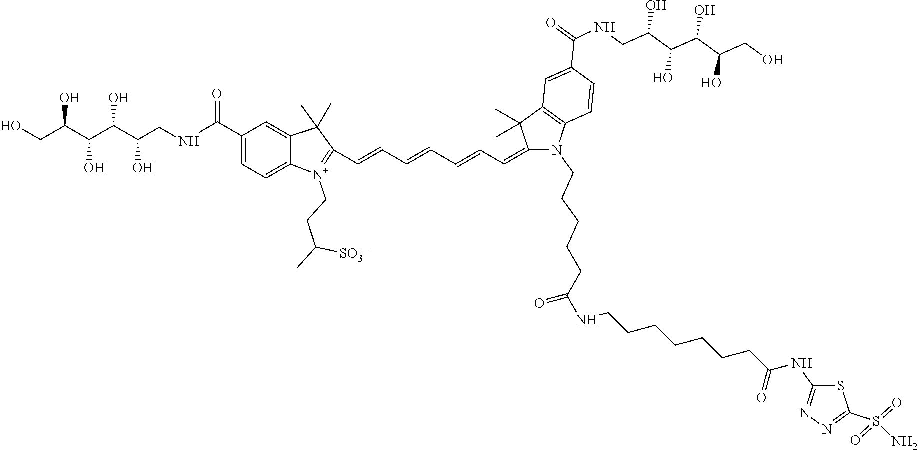
Compound 2

The present invention is also directed to methods for synthesizing the compounds of formula (I) prepared as illustrated in the following of the description. The compounds of the invention are useful as imaging agents in the detection of tumors in both humans and animals. Accordingly, the invention provides the compounds of formula (I) as defined above for use as fluorescent probes for the detection and demarcation of tumor tissue during diagnostic, interventional imaging and intraoperative procedures, in particular wherein said tumor is a tumor showing an increased or variable expression of CA-IX. Preferably the imaged subject is a human.

The invention also provides a compound of formula (I) for use as fluorescent probe as defined above, wherein the detection and demarcation of the tumor tissue is carried out under NIR radiation.

Preferably, the fluorescent probes of the invention are able to selectively target cells or tissues of tumors expressing CA-IX selected from brain cancer, breast cancer, head and neck cancer, ovarian cancer, prostate cancer, esophageal cancer, skin cancer, gastric cancer, pancreatic cancer, bladder cancer, oral cancer, lung cancer, renal cancer, uterine cancer, thyroid cancer, liver cancer, and colorectal cancer, including both primary tumors and regional and distant metastases.

The probes of the invention are able to identify in vivo a diseased tissue in a subject in need thereof. This can be accomplished by administering a compound of formula (I) as defined above and irradiating in vivo a body of the subject in need containing the diseased tissue with light having at least one excitation wavelength in the NIR range from about 650 nm to about 850 nm. Fluorescence emanating from said administered compound which has specifically bound to the diseased tissue in the body part in response to the at least one excitation wavelength is directly viewed to determine location and/or surface area of the diseased tissue in the subject.

In detail, the present invention also provides the compounds of formula (I) for use in a method for detecting the possible presence of a disease in a subject comprising the steps of:
administering to a subject in need of diagnosis an amount of a compound of formula (I) as defined above for a time and under conditions that allow for binding said compound to CA-IX expressing cells;
measuring a signal from said compound in a biological sample;
comparing said signal with at least one control data set comprising signals from the compound of formula (I) contacted with a biological sample that does not comprise the target cell type, for indicating the possible presence of the disease.

The present invention further provides the compounds of formula (I) as imaging agents for use in a method of imaging tissues and cells comprising the steps of:
contacting the tissues or cells with a compound of formula (I) as defined above;
irradiating the tissues or cells at a wavelength absorbed by the imaging agent;
detecting a near-infrared emission using a fluorescent camera.

In particular, in a preferred embodiment the invention provides a compound of formula (I) for use in a method of performing imaging guided surgery on a subject comprising the steps of:
administering a composition comprising a compound of formula (I) as defined above under conditions and for a time sufficient for the compound to accumulate at a given surgical site;
illuminating and visualizing said compound using near-infrared light;
performing surgical resection of the areas that fluoresce upon excitation by the near-infrared light.

A further aspect of this invention relates to a pharmaceutical composition comprising a fluorescent probe of formula (I) as defined above, or a salt thereof, and one or more pharmaceutically acceptable adjuvants, excipients or diluents.

Another aspect of this invention relates to a diagnostic kit comprising a compound of formula (I) as defined above. In addition, the kit can contain additional adjuvants for implementing a biomedical optical imaging application. These adjuvants are, for example, suitable buffers, vessels, detection reagents or directions for use. The kit preferably contains all materials for an intravenous administration of the compounds of the invention.

An effective amount of a compound of the invention may be administered by different routes prior to the imaging procedure, based on the disease to be treated and the location of the suspected disease to be diagnosed.

For instance, it can be administered to the organ or tissue to be imaged by a topical route, e.g. transdermally, an enteral route, e.g. orally, or a parenteral route, e.g. intradermally, subcutaneously, intramuscularly, intraperitoneally or intravenously.

In some embodiments the compounds of the invention can be administered by topically spraying or nebulizing pharmaceutical compositions comprising them and/or specifically formulated for that use.

The compositions are administered in doses effective to achieve the desired optical image of a tumor, tissue or organ, which can vary widely, depending on the compound used, the tissue subjected to the imaging procedure, the imaging equipment being used and the like.

The exact concentration of the imaging agents in a pharmaceutical composition is dependent upon the experimental conditions and the desired results, but typically may range between 1 pM to 0.1 mM. The optimal concentration is determined by systematic variation until satisfactory results with minimal background fluorescence are obtained. Once administered, the imaging agents of the invention are exposed to a light, or other form of energy, which can pass through a tissue layer. Preferably the radiation wavelength or waveband matches the excitation wavelength or waveband of the photosensitizing agent and has low absorption by the non-target cells and the rest of the subject, including blood proteins. Typically, the optical signal is detectable either by observation or instrumentally and its response is related to the fluorescence or light intensity, distribution and lifetime.

The preparation of the compounds of formula (I), as such or in the form of pharmaceutically acceptable salts, represents a further aspect of the invention. The compounds of the invention can be prepared for instance according to the methods described in the following sections and in the experimental part. A general teaching about the preparation of cyanine scaffold can be found in Mujumdar R. B. et al., Bioconjugate Chem. 1993, 4(2), 105-111, which relates to the synthesis and labeling of sulfoindocyanine dyes.

However, in some cases, because of the presence of different functional moieties such as carboxylic acid or amide groups the cyanines of the present invention, the use of protecting groups was necessary to direct the reactions on the desired functional group.

Generally, special attention is required when manipulating the cyanines at the strong pH and temperature conditions in order to avoid the removal of the protecting groups, since the stability of the polymethine scaffold can be compromised in some cases, with severe degradation of the dyes. Contrary to what was expected, the compounds of the invention have been found very stable at basic pH (i.e. at about pH 11-12) and none or negligible degradation has been observed during the removal of the protecting groups.

### EXPERIMENTAL PART

The invention and its particular embodiments described in the following part are only exemplary and not to be regarded as a limitation of the present invention: they show how the present invention can be carried out and are meant to be illustrative without limiting the scope of the invention.

### Materials and Equipment

All commercially available reagents used in the synthesis were obtained from Sigma Aldrich and TCI and used without further purification. Reference compound 1 (HF-1) was prepared as described in Mahalingam S. M. et al., Bioconjugate Chem 2018, 29, 3320-3331, while reference compounds 2 and 3 were prepared as described respectively in WO2021/259899 and in WO2020/229438.

All the reactions were monitored by HPLC (Agilent mod. 1100/1200) and HPLC-MS (Agilent mod. 1260, Quadrupole LC/MS Mod. 6120) equipped with an absorption detector set at different wavelengths (Column: YMC-Triart Phenyl, 250×4.6 mm/S-5 μm/12 nm), eluents: 0.1% ammonium acetate and acetonitrile.

Flash chromatographic purifications were performed on an automated purification system (CombiFlash® Rf+, Teledyne ISCO), using pre-packed silica C18 cartridges (Biotage® SNAP or SFÄR), generally eluting with water/acetonitrile gradient.

A dual-beam UV-VIS spectrophotometer (Lambda 40, Perkin Elmer) was used to determine the absorbance (Abs) of the compounds of the invention. Emission/excitation (Em/Ex) spectra were carried out on a spectrofluorometer (FluoroLog-3 1IHR-320, Horiba Jobin Yvon) equipped with an F-3018 integrating sphere accessory. The measurements were performed using an excitation wavelength at maximum absorbance of different dyes, and the sample was excited with a 450 W Xenon light Source. Detection was performed by photomultiplier tubes (PMT-NIR) cooled detector or by TBX-04 detector. Dye solutions were carefully prepared to have an absorbance lower than 0.1 (optical densities) to minimize re-absorption phenomena.

Human colon adenocarcinoma HT-29 cells (ATCC) used for the cellular binding experiments were cultured in McCoy's 5A medium (Sigma-Aldrich), supplemented with 10% HyClone Fetal Clone III (Euroclone), 2 mM L-glutamine (Sigma-Aldrich), 100 IU/mL penicillin, 0.1 mg/mL streptomycin, 0.25 μg/mL amphotericin B (Antibiotic-Antimycotic solution, Life Technologies) and cells were grown at 37° C. in humidified atmosphere enriched with 5% CO₂. DPBS without MgCl₂ and CaCl₂) (Sigma-Aldrich) was used for cell rinsing.

### List of Abbreviations

DCM dichloromethane
DMF N,N-Dimethylformamide
DNSA 5-(dimethylamino)naphtalene-1-sulfonamide; dansylamide
TEA triethylamine
HPLC High performance liquid chromatography
HSA Human serum albumin
NMR Nuclear magnetic resonance
t_{R} Retention time (HPLC)
RT Room temperature
CV Column volume
DMSO Dimethyl sulfoxide
PBS Phosphate buffered saline
DPBS Dulbecco's phosphate buffer saline
FMOC 9-Fluorenylmethoxycarbonyl
C8-AZA 8-amino-N-(5-sulfamoyl-1,3,4-thiadiazol-2-yl)octanamide
NMM N-methylmorpholine
HBTU O-Benzotriazol-1-yl-N,N,N′,N′-tetramethyluronium hexafluorophosphate
HATU 1-[Bis(dimethylamino)methylene]-1H-1,2,3-triazolo[4,5-b]pyridinium 3-oxide hexafluorophosphate
TBTU 2-(1H-Benzotriazole-1-yl)-1,1,3,3-tetramethyluronium tetrafluoroborate
TSTU O—(N-Succinimidyl)-1,1,3,3-tetramethyluronium tetrafluoroborate
COMU (1-Cyano-2-ethoxy-2-oxoethylidenaminooxy)dimethylamino-morpholino-carbenium hexafluorophosphate

### Example 1: Synthesis of Compound 1

### Preparation of the Intermediate Compound C8-AZA

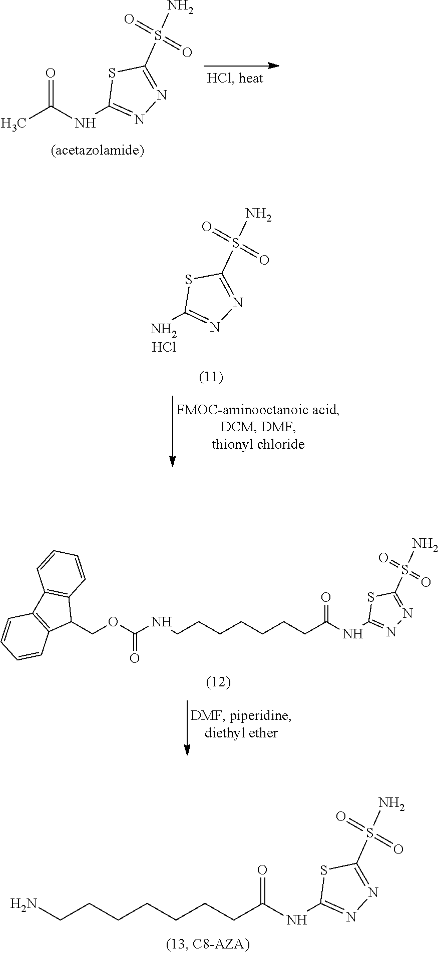 [SMILES: CC(=O)Nc1nnc(S(N)(=O)=O)s1 ; Nc1nnc(S(N)(=O)=O)s1 ; Cl ; NS(=O)(=O)c1nnc(NC(=O)CCCCCCCNC(=O)OCC2c3ccccc3-c3ccccc32)s1 ; NCCCCCCCC(=O)Nc1nnc(S(N)(=O)=O)s1]

In a round bottom flask acetazolamide (1103.56 mg, 4.96 mmol) was suspended in 1 M HCl (26 mL) and heated up to reflux for 1.5 hour, obtaining a clear solution. The mixture was concentrated under reduced pressure and purified by flash chromatography on a pre-packed C18 silica column (Biotage® Sfär C18 60 g) with a water-acetonitrile gradient (4 CV at 0% ACN, linear gradient from 0 to 100% ACN in 2 CV). Fractions containing the pure intermediate compound 11 thus obtained (eluted with 100% water) were combined, concentrated under reduced pressure and freeze-dried, obtaining a white solid (775.24 mg, 72% yield). HPLC purity: 97.8% at 270 nm, [M+H]⁺ 181.1.

In a round bottom flask cooled in an ice bath, Fmoc-8-aminooctanoic acid (500 mg, 1.311 mmol) was suspended in anhydrous DCM (30 mL) and a small amount of anhydrous DMF (20 μL, 0.262 mmol) was added. Then, thionyl chloride (2.85 mL, 39.33 mmol) was slowly dropped. The mixture was stirred at room temperature for about 30 minutes, observing complete dissolution. Solvents and residual thionyl chloride were concentrated under reduced pressure. The yellow crude oil was immediately dissolved in a solution of compound 11 (283 mg, 1.311 mmol) and anhydrous pyridine (211 μL, 2.62 mmol) in anhydrous DMF (10 mL) and stirred at room temperature for 1.5 hour. The solution was then concentrated under reduced pressure and the intermediate compound 12 was precipitated with DCM. The white solid was filtered and dried under vacuum at 30° C. overnight (294.25 mg, 41% yield). HPLC purity: 93% at 270 nm and 254 nm, MS: [M+H]⁺ 544.2.

Compound 12 (294.25 mg, 0.541 mmol) was then dissolved in a round bottom flask in anhydrous DMF (5.8 mL) and piperidine (1.16 mL) was added. After 30 minutes cold diethyl ether (100 mL) was added; the solid was then filtered, dissolved in methanol, the solution was concentrated to solid residue and further treated in diethyl ether (100 mL). Finally, the intermediate compound 13 (C8-AZA) was recovered as a white powder which was dried under vacuum at room temperature overnight (163.56 mg, 94% yield). HPLC purity: 96.6% at 270 nm and 96.7% at 254 nm, MS: [M+H]⁺ 322.2.

### Preparation of the Intermediate Compound (15)

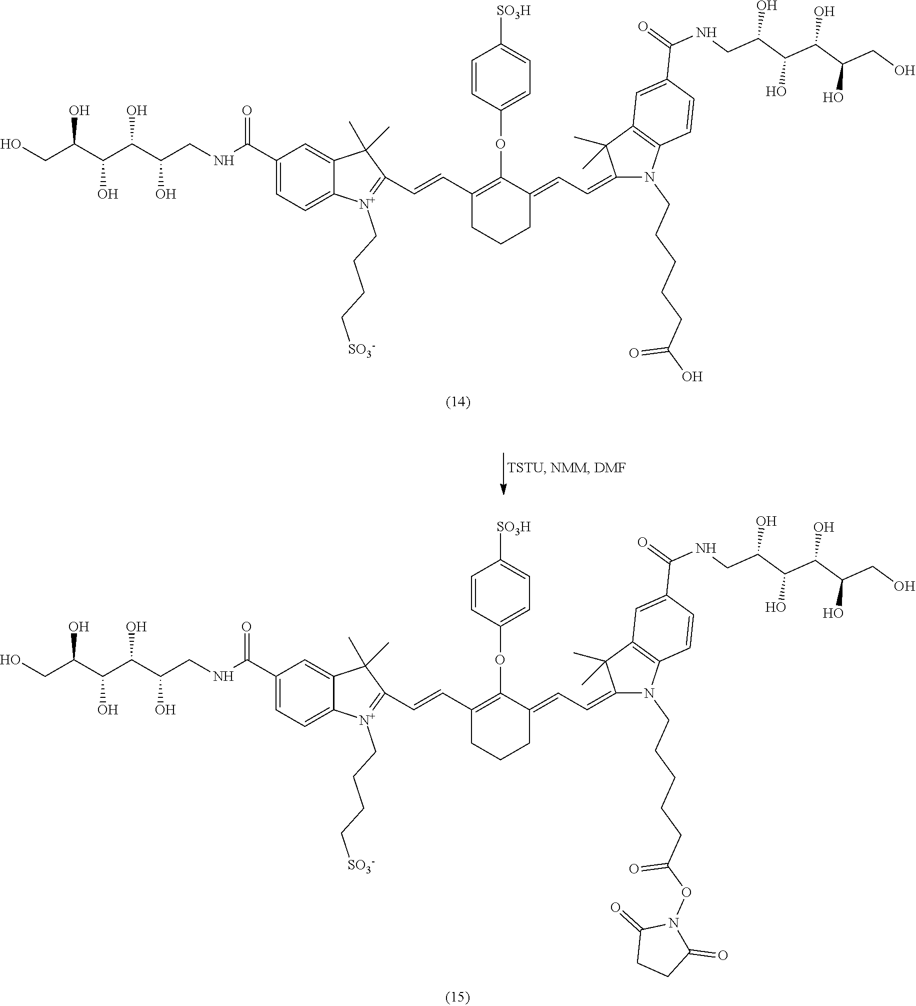

Compound 14 (70.1 mg, 0.0547 mmol, prepared as described in WO2021/259899) was dissolved in anhydrous DMF (8 mL). TSTU (32.9 mg, 0.109 mmol) and NMM (18 μL, 0.164 mmol) were added at RT and the dark green solution was stirred at RT in the dark for 1 hour.

Then, the solution was quickly dropped into cold ethyl acetate (100 mL) under vigorous stirring in ice bath and the suspension was let at −20° C. for 30 minutes and then filtered. The dark green solid was washed with ethyl acetate (2×10 mL), dried under nitrogen flow and used in the following step with no further purification (intermediate 15 conversion: 90% at 780 nm).

### Synthesis of Compound 1

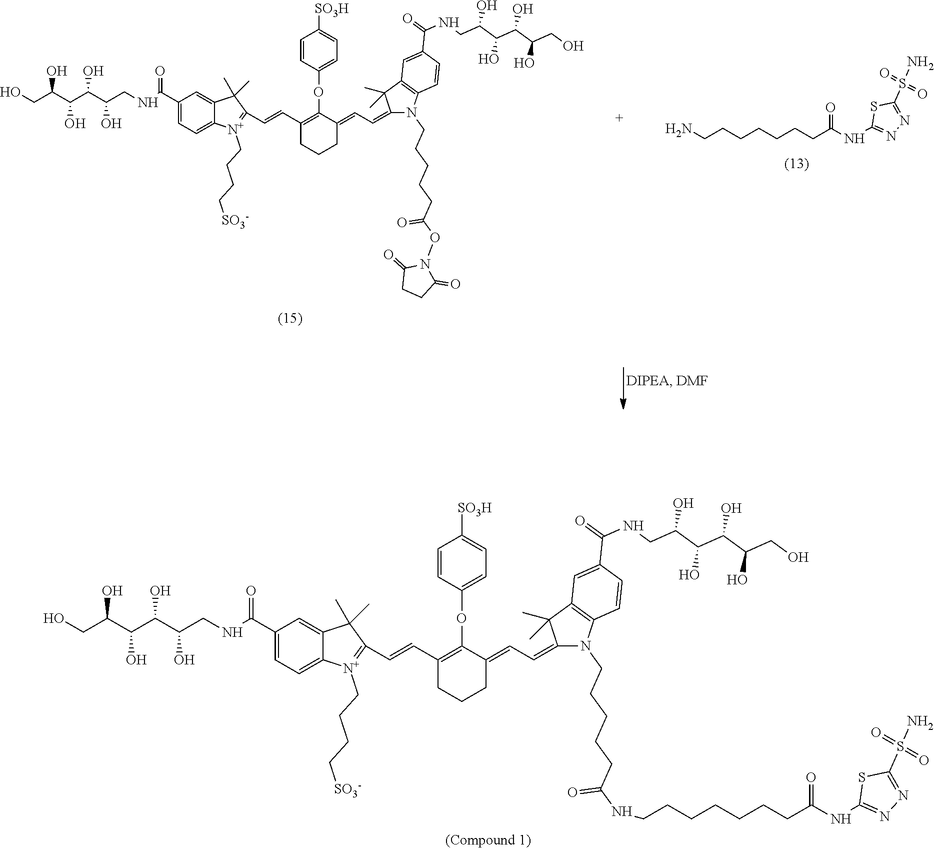

The solid of compound 15 was dissolved in anhydrous DMF (8 mL) and then a solution of compound 13 (21.1 mg, 0.0657 mmol) and DIPEA (48 μL, 0.273 mmol) in anhydrous DMF (1 mL) was added. The solution was stirred at RT in the dark for 1 hour. An additional 4 mg of compound 13 were added and the solution was stirred for other 30 minutes. The dark green solution was then dropped into cold ethyl acetate (150 mL), the so obtained precipitate was dissolved in water and purified by flash chromatography on a pre-packed C18 silica column (Biotage SFÄR 30 g) with a water-acetonitrile gradient (2 CV of 100% water, from 0 to 25% ACN in 2.5 CV, 5 CV at 25% ACN and 3 CV at 100% ACN). Fractions containing the pure product (eluted with 25% ACN) were combined, concentrated under reduced pressure and freeze-dried, obtaining 53.3 mg of a dark green solid (HPLC purity: 97.5% at 780 nm and 98.4% at 254 nm, 61% yield).

### Example 2: Synthesis of Compound 2

### Preparation of the Intermediate Compound (17)

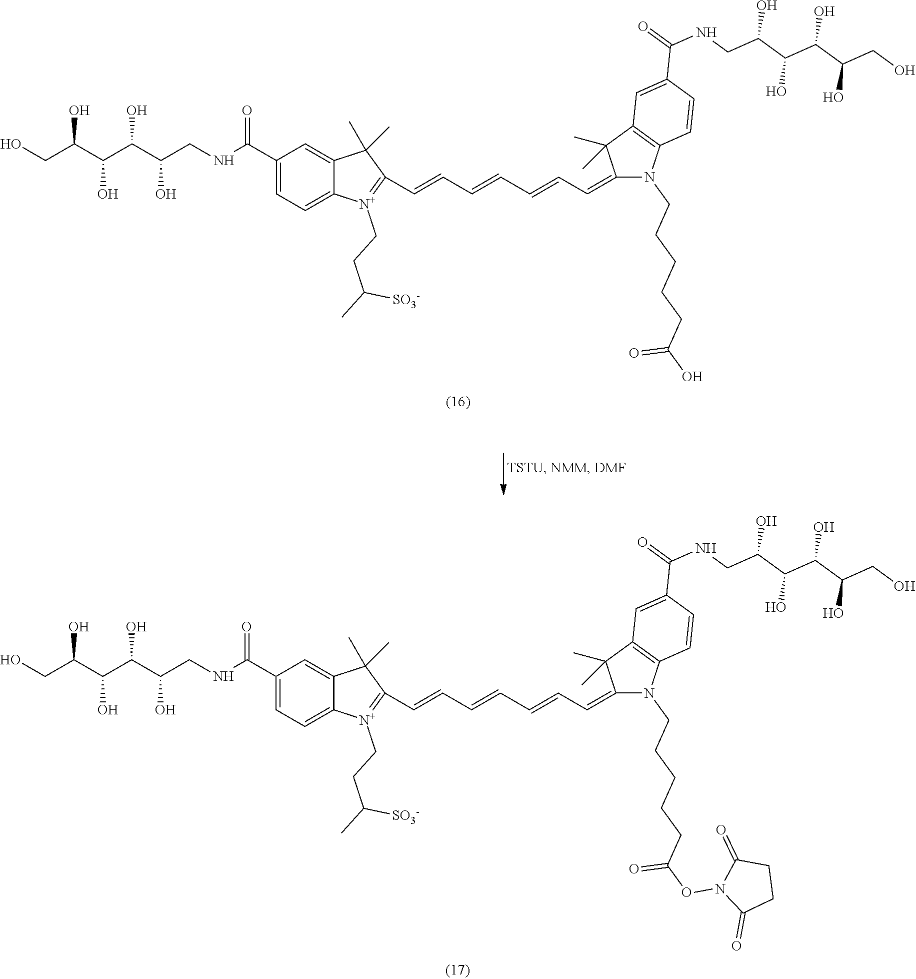

Compound 16 (50.3 mg, 0.0472 mmol, prepared as described in WO2020/229438) was dissolved in anhydrous DMF (5 mL). TSTU (21.3 mg, 0.0708 mmol) and NMM (12 μL, 0.108 mmol) were added at RT and the dark green solution was stirred at RT in the dark for 1 hour.

Then, the solution was quickly dropped into cold ethyl acetate (25 mL) under vigorous stirring in ice bath and the suspension was let at −10° C. for 22 hours and then filtered. The dark green solid was washed with ethyl acetate (2×10 mL), dried under nitrogen flow, dissolved in few mL of water and freeze-dried. The solid was then used in the following step with no further purification (intermediate 17 conversion: 84% at 756 nm).

### Synthesis of Compound 2

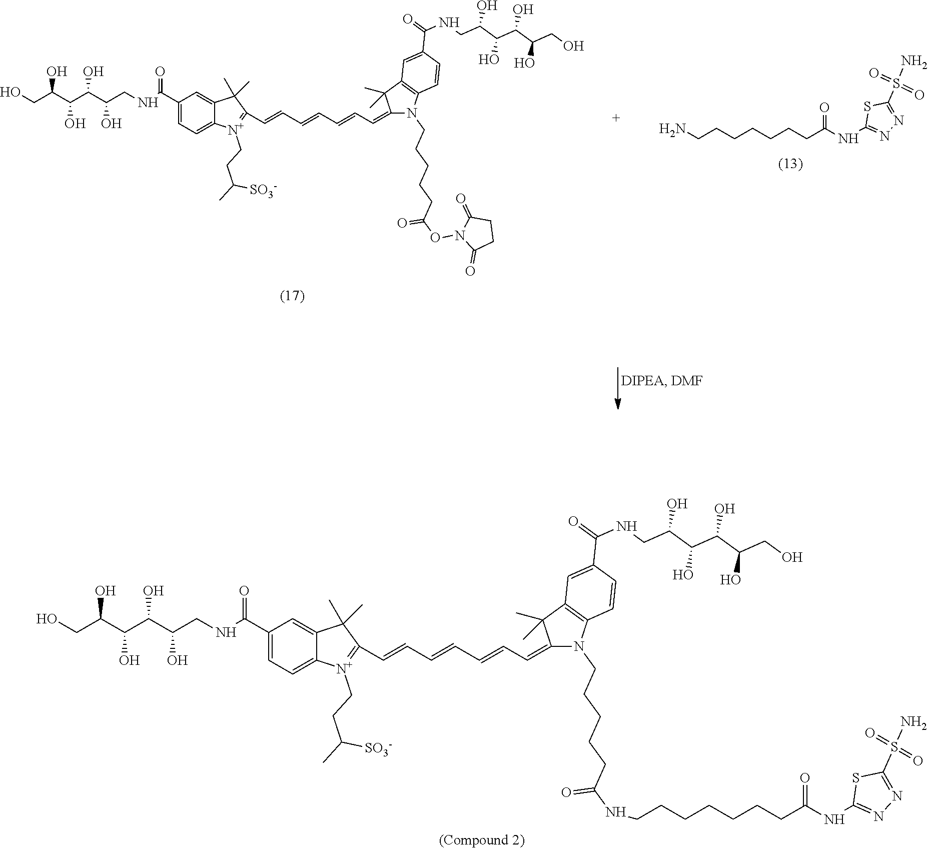

Compound 17 (58 mg, 0.041 mmol) was dissolved in anhydrous DMF (6 mL) and then a solution of compound 13 (10 mg, 0.0311 mmol) and DIPEA (32 μL, 0.196 mmol) in anhydrous DMF (2 mL) was added. The solution was stirred at RT in the dark for 2.5 hour. The dark green solution was then dropped into cold diethyl ether (30 mL), let few hours at −20° C. and then filtered. The so obtained precipitate was dissolved in water and purified by flash chromatography three times on a pre-packed C18 silica column (Biotage SFÄR 30 g) with a water-acetonitrile gradient. Fractions containing the pure product (eluted with 35% ACN) were combined, concentrated under reduced pressure and freeze-dried, obtaining 39 mg of a dark green solid (HPLC purity: 97% at 756 nm and 99% at 254 nm, 92% yield vs C8-AZA).

### Example 3: Optical Characterization

The compounds of the invention were characterized in terms of their optical properties in vitro in aqueous medium (i.e., water/PBS pH 7.4) and in a clinical chemistry control serum (Seronorm, Sero SA), mimicking the chemical composition and optical properties of human serum. All solutions were freshly prepared. In particular, the compounds of the invention are characterized by absorption maxima comprised in the range from about 750 nm to 780 nm.

The excitation and emission maxima in PBS and Seronorm and the molar extinction coefficient (ε) in water of representative compounds of formula (I) are shown in Table I in comparison with the known CA-IX targeting fluorescent probe named Hypoxyfluor-1 (HF-1, for instance disclosed in Bioconjugate Chem 2018, 29, 3320-3331). The compounds of the invention displayed a red shift in absorption and emission in Seronorm with respect to PBS due to some interaction of the probes with serum proteins. Moreover, the extinction coefficients were higher than 200,000 mM⁻¹ cm⁻¹ for all compounds.

TABLE I
Excitation/Emission maxima and molar extinction coefficient (ε) of
representative compounds of formula (I) and Reference 1

| | Max Ex/Em | | |
|---|---|---|---|
| | (nm, PBS | Max Ex/Em | ε |
| | pH 7.4) | (nm, Seronorm) | (mM⁻¹cm⁻¹) |
| HF-1 (Reference 1) | 772/798 | 792/808 | 255,000 |
| Compound 1 | 782/806 | 792/812 | 250,000 |
| Compound 2 | 758/788 | 762/790 | 250,000 |

The compounds of the invention are characterized by emission maxima which are only slightly variated when measured in Seronorm compared to PBS, indicating a moderate interaction with serum proteins.

### Example 4: Receptor Binding Affinity

### Affinity to Carbonic Anhydrase Catalytic Site

The binding affinity of the compounds of formula (I) to Carbonic Anhydrase (CA) catalytic site was determined to assess the targeting efficacy of the molecular vectors, even after labeling the targeting ligand with the fluorescent portion of the probe.

In detail, the receptor affinity of representative probes bearing the acetazolamide moiety as targeting ligand to a CA catalytic site was determined by testing their binding to a recombinant bovine CA-II enzyme (bCAII). Such enzyme in fact is highly available and has a catalytic site with high degree of homology in different carbonic anhydrase isoforms (Aggarwal M. et al., J Enzyme Inhib Med Chem 2013; 28(2): 267-277).

The dissociation constant (K_{D}) of compounds of formula (I) for bCAII was estimated by performing a fluorescence-based assay, exploiting the drop of fluorescence deriving from the displacement of carbonic anhydrase-bound dansylamide (DNSA) in presence of competitors and was adapted for application in 96-well plates from Wang S C et al., Biochem Mol Biol Educ. 2006, 34(5): 364-368. This assay is based on the fluorescence emission spectrum of DNSA, which displays differential peaks accordingly to state of the molecule: emission at about 570 nm when DNSA is free in solution, and emission at about 460 nm when DNSA is bound to CA. In both cases DNSA is excited at about 320 nm. Thus, excitation of carbonic anhydrase aromatic residues (Trp) at 285 nm generates an emission at 340 nm, that is quenched in presence of bound DNSA. The quenching occurs as FRET (fluorescence resonance energy transfer) to bound DNSA, which is in turn excited, emitting at 460 nm. Unbound DNSA, which is not in proximity of the excited Trp, is unaffected by this phenomenon and does not generate any background disturbing fluorescence. Molecules that bind to CA in the same binding site of DNSA, such as sulfonamides or derivatives, competitively displace DNSA from CA. The degree of displacement is function of the affinity of competitors and can be followed through the drop of fluorescence at 460 nm.

In detail, 5-(Dimethylamino)-1-naphthalenesulfonamide (dansylamide, DNSA, Sigma-Aldrich 218898) was reconstituted in 20 mM Tris pH 8, aliquoted and stored at −20° C.; bCAII (recombinant, expressed in E. Coli, Sigma-Aldrich) was divided in aliquots and stored at −20° C.). Scalar concentrations of compounds of formula (I) (compound 1: range 0-9.5 μM; compound 2: range 0-5 μM), or reference compounds (reference 2: range 0-21.1 μM; reference 3: range 0-22.5 μM), or acetazolamide, as internal control (range 0-22.5 μM), were incubated with 0.25 μM of bovine CA-II and 5 μM DNSA in 20 mM Tris pH 8.

DNSA fluorescence emission at 462 nm from each well was recorded after excitation at 285 nm (Trp wavelength of excitation) and consequent selective excitation of DNSA bound to bCAII by FRET. Fractional saturation of bCAII was experimentally determined through normalization of fluorescence data between 1 (no competing compounds) and 0 (maximal DNSA displacement by presence of binding competitors), according to the following equation:

$$r = \frac{{FL}_{obs} - {FL}_{min}}{{FL}_{max} - {FL}_{min}}$$

where r is fractional saturation, F_{Lobs} is the recorded fluorescence intensity, FLₘᵢₙ is the fluorescence intensity recorded at maximal concentration of compounds, and FLₘₐₓ is fluorescence recorded in absence of compounds.

The K_{D} of CA-binding compounds (=KD_{comp}) was then derived by mathematical fitting of experimental data according to the equation:

$$r = \frac{1}{1 + \frac{K {D}_{D N S A}}{[ D N S A ]} * ( 1 + \frac{[ comp ]}{K {D}_{c o m p}} )}$$

where r is fractional saturation (calculated from experimental data as described above), [DNSA] is DNSA nanomolar concentration, KD_{DNSA} is the dissociation constant at equilibrium of DNSA (previously experimentally determined=169.6 nM), and [comp] is the nanomolar concentration of the CA-targeting compound under analysis. Mathematical fitting was performed using Microsoft Excel Solver. Acetazolamide was included in every test as internal control of the assay (acetazolamide K_{D}=29.6±2.6 nM, previously estimated).

All the compounds were tested at least in duplicate in independent experiments. The resulting K_{D} of the experiments are summarized in Table II (average of independent experiments, with standard deviations) and compared with the corresponding result obtained for reference compounds and the unlabeled acetazolamide.

The tested probes showed a very high affinity to the carbonic anhydrase receptor, displaying K_{D} values lower than 15 nM, about 2-5 times lower than acetazolamide and respectively 4 and 5 times lower of the corresponding reference compound bearing the same cyanine dye but conjugated with a different CA-IX targeting moiety (comparing compound 1 with Reference 2 and compound 2 with Reference 3).

TABLE II
Binding affinity to the CA-II receptor of representative
compounds of formula (I) compared to the unlabeled
acetazolamide and to reference compounds 2, 3.

| | CA-II receptor affinity |
|---|---|
| | (K_{D}, nM) |
| Acetazolamide | 29.6 ± 2.6 |
| Reference 2 | 55.8 ± 1.1 |
| Reference 3 | 30.2 ± 1.0 |
| Compound 1 | 13.5 ± 1.6 |
| Compound 2 | 6.1 ± 1.6 |

### Cellular Binding Assessment

To further confirm these results showing improved binding affinity, the binding of the probes to CA-IX was assessed by using an in vitro model with the human colorectal adenocarcinoma cell line HT-29 (ATCC). In fact, by both western blot and flow cytometry, HT-29 cells were demonstrated to constitutively express high levels of CA-IX under normoxic conditions. Indeed, by using the anti-human CA-IX antibody MAB2188 (R&D Systems) and the DAKO QUIFIKIT, according to the instruction of the datasheet, HT-29 cells were estimated to express about 150,000 CA-IX molecules per cell. Thus, HT-29 cells were used to measure the binding of all compounds to CA-IX, expressed in its physiological environment at the cell-surface. Briefly, HT-29 cells were detached with StemPro® Accutase® Cell Dissociation Reagent (Life Technologies), collected in DPBS and counted. At least 2·10⁵ HT-29 cells were placed in 1.5 mL tubes on ice and resuspended in 50-100 μL of cold FACS buffer (eBioscience™ Flow Cytometry Staining Buffer, Invitrogen) containing scalar concentrations of compounds (HF-1, reference 2, and compound 1: range 0-20 μM; reference 3: range 0-24 μM; compound 2: range 0-10 μM). After incubation (1 hour and 30 min on ice in the dark), cells were rinsed 3 times in 300 μL of cold FACS buffer by centrifugation (5 min, 4° C., 350 RCF), discarding the supernatant. Cell pellets were then resuspended in 200 μL of cold FACS buffer (or in an adequate volume in order to obtain no more than 1000 recorded events per μL during FACS analysis) and analyzed with the flow cytometer Accuri™ C6 (BD Biosciences). The mean cell-associated fluorescence was plotted against concentration and the dissociation constant at equilibrium (K_{D}) was inferred by mathematical fitting with GraphPad Prism v.9 software, according to the following equation:

$$Y = \frac{{B}_{max} * X}{X + {K}_{D}} + N S * X + background$$

where Bₘₐₓ is the maximum specific binding in the same units as Y; K_{D} is the equilibrium dissociation constant, in the same units as X; NS is the slope of nonspecific binding in Y units divided by X units; and background is the amount of nonspecific binding with no added Compound.

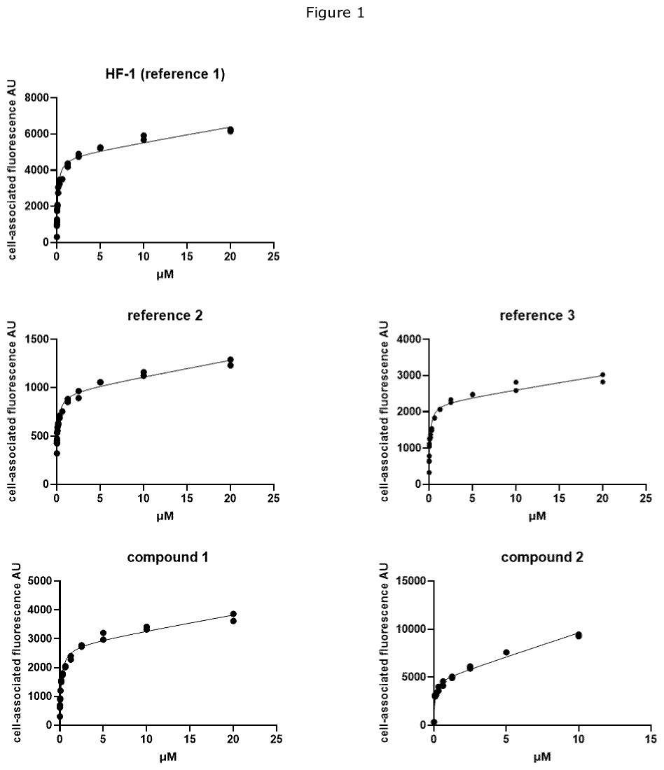

All compounds were analyzed in at least three independent experiments. The calculated K_{D} of the representative compounds 1 and 2 of the invention is summarized in Table III, in comparison to the one obtained respectively for reference compounds 2 and 3; representative binding curves are shown in FIG. 1

TABLE III
Binding affinity to CA-IX expressing HT-29 cells of
representative compounds 1 and 2 of the invention in
comparison with the reference compounds 1, 2 and 3.

| | CA-IX receptor affinity |
|---|---|
| | (K_{D}, nM to HT-29 cells) |
| Reference 1 | 115 ± 8 |
| Reference 2 | 349 ± 49 |
| Reference 3 | 251 ± 44 |
| Compound 1 | 174 ± 15 |
| Compound 2 | 58 ± 9 |

This assay confirmed that compounds of the invention bearing a C8-AZA moiety are endowed of a high affinity to CA-IX expressing cells. In detail, compound 1 of the invention showed a remarkable improvement in affinity to the target, with a K_{D} 2 fold lower than the corresponding Reference compound 2, while compound 2 of the invention showed a K_{D} more than 4 fold lower than the corresponding Reference compound 3. Compound 2 also showed a K_{D} 2 fold lower than Reference 1 (HF-1), while compound 1 is rather comparable to Reference 1.

### Cellular Specificity

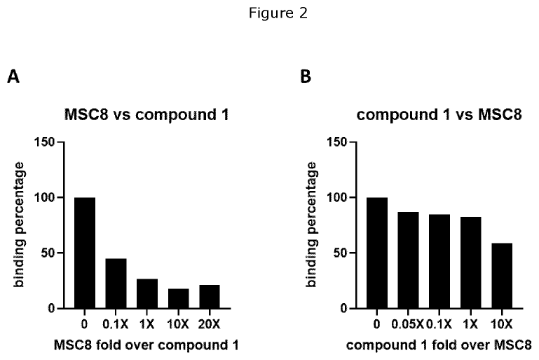

For compound 1, an additional in vitro binding test was conducted to assess the CA-IX targeting specificity, by using the fully human recombinant MSC8 antibody, which has been demonstrated to selectively recognize the isoform IX of human CAs and block CA-IX enzymatic activity (WO2011/139375). HT-29 cells were co-incubated, on ice in suspension, with compound 1, at the established concentration of 0.125 μM, and different concentrations of MSC8 (0.1×, 1×, 10× and 20× with respect to compound 1, i.e. 0.0125/0.125/1.25/2.5 μM); in addition, HT-29 cells were incubated with either compound 1 alone, or each concentration of MSC8 alone as well. The resulting binding of both compound 1 and MSC8 was then recorded as cell-associated fluorescence intensity, using for MSC8 a secondary antibody labelled with Alexa Fluor™ 488 (ThermoFischer), and compared to the one resulting from both compounds alone, taken as 100%. Indeed, MSC8 reduced in a dose-dependent manner the binding of compound 1 to HT-29 cells, indicating that it prevented its access to the catalytic pocket of CA-IX. Viceversa, also compound 1 reduced, in a dose-dependent manner, the binding of MSC8, with an effect visible especially at 10× concentration (i.e. compound 1 at 0.125 μM and MSC8 0.0125 μM). Results are shown in FIG. 2.

### Example 5: Cell Uptake

The receptor-mediated uptake, i.e. whether the uptake of the probes was mediated by binding to the target receptor CA-IX, was analyzed by performing competition experiments. Compounds were administered to cells (1 μM, 2 hours at 37° C.) in presence or absence of 100× excess of acetazolamide, competing with the co-administered compounds for CA-IX binding site. In the above conditions, the cell-associated fluorescence for each probe decreased to the values reported in Table IV, indicating that most of the recorded cellular uptake relied on CA-IX targeting. In particular, for both representative compounds 1 and 2, the cell-associated fluorescence recorded in flow cytometry experiments was demonstrated to rely for at least 90% on cell surface CA-IX bound probe, with the remaining about 10% due to non-specific cellular uptake.

TABLE IV
Residual cellular uptake of representative
compounds of formula (I)

| | % uptake in presence of 100x excess acetazolamide |
|---|---|
| Compound 1 | 6.5 ± 2.2 |
| Compound 2 | 11.3 ± 1.7 |

### Example 6: Binding to Albumin

The affinity of the probes for human serum albumin (HSA) was determined to assess the influence of structural features on their albumin binding properties. Briefly, HSA (A9511, Sigma-Aldrich) was prepared in a 0.5 M stock solution in PBS and used to obtain a series of dilutions in PBS (0, 9.52·10⁻⁷, 4.76·10⁻⁶, 9.52·10⁻⁶, 1.90·10⁻⁵, 3.81·10⁻⁵, 5.71·10⁻⁵, 9.52·10⁻⁵, 1.43·10⁻⁴, 1.90·10⁻⁴, 2.86·10⁻⁴, 3.81·10⁻⁴ M), in presence of 4.76 μM of the CA-IX-targeting probe (prepared from a 50 μM stock solution in PBS), in a total volume of 0.525 mL. The samples were centrifuged (10000 g for 30 min at 25° C.) in a Microcon device (10 KDa MW cut off, Amicon Ultra-0.5 Centrifugal Filter Unit with Ultracel-10 membrane) and the absorbance measurements of the filtrates were obtained with the spectrophotometer at the maximum absorbance wavelength of the fluorophore.

Data analysis was performed by fitting the raw data, using Microsoft Excel Solver, with the equation:

$$\frac{Δ A}{b} = \frac{( Δ ε \cdot {K}_{R L} \cdot [ L ] \cdot {R}_{t} )}{{K}_{R L} \cdot [ L ] + 1}$$

where ΔA/b=Absorbance measured (b=1 cm), K_{RL}=K_{A} calculated by fitting, Δε·Rₜ are calculated by fitting, and [L]=HSA concentration. ΔA/b values were experimentally obtained by subtracting the absorbance of “0 HSA sample” to the absorbance of each other sample. The obtained K_{A} of compounds for HSA are reported in Table V.

TABLE V
Affinity for human serum albumin (K_{A})

| | HSA affinity (K_{A}, M⁻¹) |
|---|---|
| Reference 1 (HF-1) | 156,300 |
| Reference 2 | 14,300 |
| Reference 3 | 41,700 |
| Compound 1 | 12,500 |
| Compound 2 | 9,500 |

Of note, representative compounds 1 and 2 showed a lower binding to HSA compared to HF-1, similarly to reference compounds 2 and 3, which respectively share the same parent dyes, and consequently they also show a better expected bioavailability (K_{A} in the 10⁴ M⁻¹ order of magnitude or lower).

### Example 7: Tumor Imaging Efficacy

To evaluate the in vivo efficacy of the compounds of the invention, a hypoxic HT-29 colorectal tumor, subcutaneously induced, was used as an animal model due to the well-known hypoxic tumor microenvironment. HT-29 cells were collected and washed once with serum-free medium. Five million HT-29 cells were suspended in 0.1 mL serum-free medium and injected subcutaneously in the right flank of 5 weeks old mice. Tumor development was followed by caliper measurement twice a week, from 7 days after inoculation. Tumor volume was calculated according to the formula: (L*W2)/2, where L and W are the maximum length and width of the tumor.

Optical Imaging experiments on HT-29 tumor bearing animals were performed after administration of Reference 1, 2 and 3, and compounds 1 and 2, all at a dose of 1 nmol/mouse in an administration volume of 0.1 mL (corresponding to about 5 mL/kg, considering a 20 g-mouse) at an injection rate of about 1 mL/min, by the IVIS Spectrum system when the tumor reached at least 200 mm³ volume.
For each group, in vivo OI experiments were performed at 0.5, 1, 2, 3, 4, 6 and 24 hours after intravenous administration of the fluorescent compounds. During imaging experiments animals were maintained under gaseous anesthesia. Regions of interest (ROIs) were drawn on the tumor and on a reference background healthy region (hind limb muscle) of the mouse for each fluorescence image at every time point to evaluate signal intensity in the tissues (expressed as Average Radiant Efficiency). The ratio between the fluorescence signal in the tumor and in the background healthy tissue (tumor-to-background ratio, TBR) was then calculated to assess the contrast.

The results are displayed in FIG. 3 and in Table VI, reporting the values of in vivo TBR from samples collected at 24 hours after administration. All the compounds of the invention and the References were tested at a dose of 1 nmol/mouse.

The compounds of the invention showed selective accumulation in the tumor and values of TBR higher than the Reference compounds at the same dose of 1 nmol/mouse. In particular they showed a TBR from 2 to 8.5 times higher than Reference 1 and from 1.5 to 2.5 times higher than the corresponding References 2 and 3.

TABLE VI
Values of in vivo TBR (mean ±
SD) at 24 hours after administration

| | TBR (mean ± SD) |
|---|---|
| Reference 1 (HF-1) | 1.8 ± 0.3 |
| Reference 2 | 3.4 ± 0.6 |
| Reference 3 | 4.7 ± 0.6 |
| Compound 1 | 5.2 ± 1.0 |
| Compound 2 | 10.1 ± 3.1 |

### Example 8: Biodistribution and Elimination

The ex vivo biodistribution of the representative compounds 1 and 2 and of the reference compounds (at 1 nmol/mouse) was evaluated in HT-29 excised tumors and tissues at the end of the in vivo OI sessions 24 hours post intravenous administration. At the end of the in vivo experiment, animals were euthanized by overdose of gaseous anesthesia followed by cervical dislocation. Tumor tissue, muscle, and other organs of interest were removed for ex vivo fluorescent signal measurements. The fluorescence signal intensity was measured by the IVIS Spectrum system. Regions of interest (ROIs) were drawn around each organ and tumor using the Living Image Software (PerkinElmer) and evaluating signal intensity in the tissues (expressed as Average Radiant Efficiency). The ratio between the fluorescence signal in the organs and tumor was then calculated to assess the contrast to hind limb muscle (representing the background).

The results are displayed in FIG. 4 (a) and in Table VII wherein it is evidenced that for the compounds of the invention, injected at the dose of 1 nmol/mouse, the fluorescence signal observed in tumor regions was remarkably higher compared to the fluorescence signal of the background (hind limb muscle), with tumor-to-background values ranging from about 13 to about 23. Conversely, it is to be noted that the Reference 1 (HF-1) at the same dose of 1 nmol/mouse showed much lower difference between fluorescent signals in tumor and in muscle (background). Moreover, compounds 1 and 2 showed a TBR about 1.5 times higher than the respective References 2 and 3.

TABLE VII
Values of ex vivo TBR (mean ± SD) from
samples collected 24 hours after administration
of compounds 1, 2 and of Reference compounds.

| TBR | Reference | Reference | Reference | | |
|---|---|---|---|---|---|
| (mean ± | 1 | 2 | 3 | Comp 1 | Comp 2 |
| SD) | (1 nmol) | (1 nmol) | (1 nmol) | (1 nmol) | (1 nmol) |
| HT-29 | 3.1 ± 0.3 | 9.2 ± 3.3 | 14.7 ± 2.3 | 13.4 ± 2.2 | 23.1 ± 6.7 |
| tumor | | | | | |

Moreover, FIG. 4 (b) and Table VIII show the accumulation and distribution of the fluorescent probes in the dissected organs, reporting the values ex vivo of muscle-to-organ ratio from samples collected at 24 hours after the administration of representative compounds 1, 2 and of the Reference compounds (1 nmol/mouse). Compounds fluorescence showed a very low concentration in most tissues, except for kidneys (probably due to renal excretion route of injected compounds) and liver. A certain uptake can be also seen in the stomach, which is known to express CAIX in the gastric mucosa.

TABLE VIII
Values of organ-to-muscle ratio (mean ± SD) 24 hours after
administration of compounds 1, 2 and of Reference compounds.

| Organ- | | | | | |
|---|---|---|---|---|---|
| to- | Reference | Reference | Reference | Comp 1 | Comp 2 |
| muscle | 1 | 2 | 3 | (1 | (1 |
| ratio | (1 nmol) | (1 nmol) | (1 nmol) | nmol) | nmol) |
| intestine | 2.2 ± 1.3 | 3.0 ± 0.3 | 2.7 ± 0.9 | 1.8 ± 0.4 | 1.6 ± 0.3 |
| kidney | 2.4 ± 0.3 | 19.7 ± 2.2 | 15.8 ± 3.4 | 6.0 ± 1.1 | 7.1 ± 1.5 |
| liver | 5.7 ± 0.2 | 6.2 ± 1.3 | 3.6 ± 1.0 | 4.2 ± 0.7 | 3.3 ± 0.4 |
| skin | 1.4 ± 0.0 | 1.7 ± 0.4 | 2.0 ± 0.5 | 1.2 ± 0.1 | 1.5 ± 0.3 |
| spleen | 1.4 ± 0.3 | 1.7 ± 0.4 | 1.2 ± 0.3 | 0.9 ± 0.2 | 1.0 ± 0.1 |
| stomach | 4.0 ± 1.7 | 6.1 ± 0.7 | 6.7 ± 1.4 | 5.4 ± 0.8 | 6.8 ± 1.3 |

### REFERENCES

1. Onda N. et al., Int J Cancer 2016, 139, 673-682
2. Tummers Q. et al., PlosOne 2015 (doi.org/10.1371/journal.pone.0129766)
3. Gioux S. et al., Mol Imaging 2010, 9(5), 237-255
4. Bunschoten A. et al., Bioconjugate Chem. 2016, 27, 1253-1258
5. Hilvo M. et al, J Biol Chem 2008; 283(41): 27799-27809
6. Wykoff et al., Cancer Res. 2000, 60, 7075-7083
7. Muz et al., Hypoxia (Auckl) 2015; 3: 83-92
8. WO2014/128258
9. McDonald et al., Am J Clin Oncol 2020; 43(7): 484-490
10. Wichert et al., Nat Chem 2015; 7(3): 241-249
11. Groves et al., Bioorg Med Chem Lett 2012; 22: 653-657
12. WO2017/161195
13. Mahalingam S. M. et al, Bioconjugate Chem 2018, 29, 3320-3331
14. T. W. Green and P. G. M. Wuts, Wiley, N.Y. 2007, 4^{th} Ed., Ch. 5
15. Aggarwal M. et al, J Enzyme Inhib Med Chem 2013, 28(2): 267-277
16. Wang S C et al., Biochem Mol Biol Educ. 2006, 34(5): 364-368
17. WO2011/139375
18. WO2021/259899
19. WO2020/229438

1. A compound of formula (I), or a pharmaceutically acceptable salt thereof, 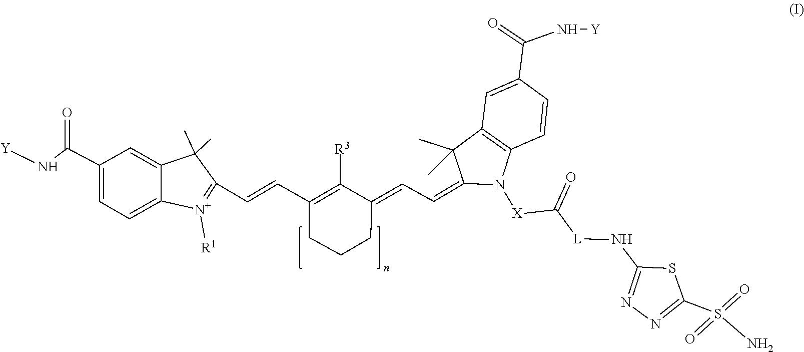
wherein
R¹ is selected from a straight or branched C₁-C₁₀ alkyl substituted by a group —SO₃H and a group of formula (II) 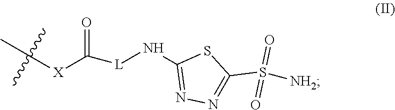
X is a straight or branched C₁-C₁₀ alkylene;
L is a bond or a linker;
each Y is independently a straight or branched C₁-C₁₀ alkyl substituted with at least two hydroxyl groups;
R³ is selected from hydrogen and a group phenyl or —O-phenyl optionally substituted with —SO₃H;
n is an integer equal to 0 or 1.

2. The compound of formula (I) according to claim 1, wherein L is a group of formula —NH—(CH₂)ₚ—CO— or a diradical of one or more moiety selected from the group consisting of an amino acid; a peptide comprising from 2 to 10 amino acids in L or D configuration; 4-aminomethylbenzoic acid; cysteic acid; a polyethylene glycol; amino-polyethylene glycol-carboxylic acid; diaminobutyric acid; and diaminopropionic acid; or it is a group -L₁-L₂- wherein L₁ is a diradical of a diamine; and L₂ is a diradical of a dicarboxylic acid; wherein p is an integer comprised between 1 and 20.

3. The compound of formula (I) according to claim 1, wherein L is a group of formula —NH—(CH₂)ₚ—CO— or a polyethylene glycol of formula —NH—(O—CH₂—CH₂)ₚ—CO— or a diradical comprising from one to five amino acids, wherein p is an integer comprised between 1 and 20.

4. The compound of formula (I) according to claim 1, wherein Y is independently selected from the group consisting of

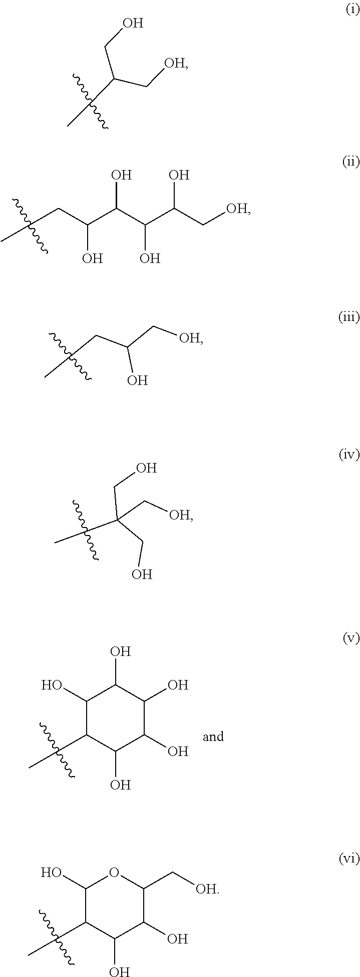 [SMILES: *C(CO)CO ; *CC(O)C(O)C(O)C(O)CO ; *CC(O)CO ; *C(CO)(CO)CO ; *C1C(O)C(O)C(O)C(O)C1O ; *C1C(O)OC(CO)C(O)C1O]

5. The compound of formula (I) according to claim 4, wherein Y is a group of formula (ii)

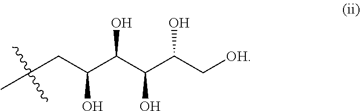 [SMILES: *C[C@H](O)[C@@H](O)[C@H](O)[C@H](O)CO]

6. The compound of formula (I) according to claim 1, wherein R¹ is a group —(CH₂)₄SO₃H or —CH₂CH₂CH(CH₃)SO₃H.

7. The compound of formula (I) according to claim 1, wherein X is a straight or branched C₁-C₆ alkylene.

8. The compound of formula (I) according to claim 1, which is represented by the formula (Ia) 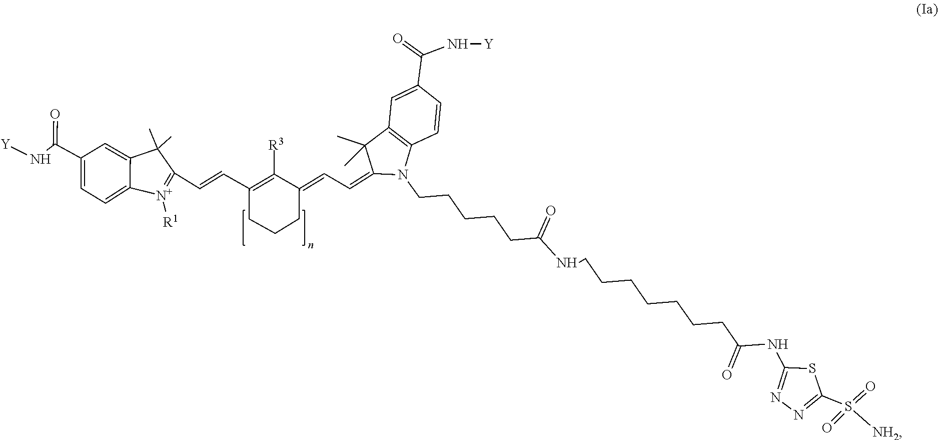
wherein R¹, R³, Y and n are as defined in claim 1.

9. The compound of formula (I) according to claim 1, which is represented by the formula (Ib) 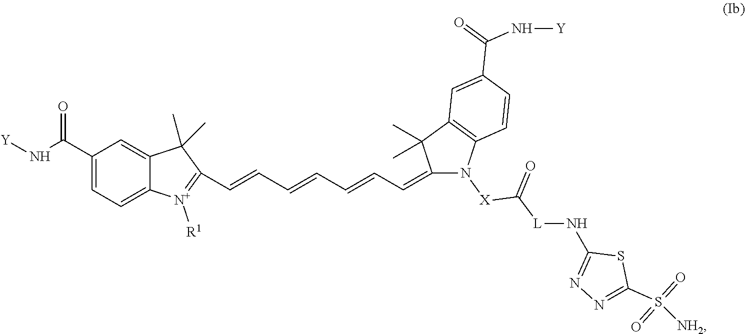
wherein R¹, X, Y and L are as defined in claim 1.

10. The compound of formula (I) according to claim 1, which is represented by the formula (Ic) 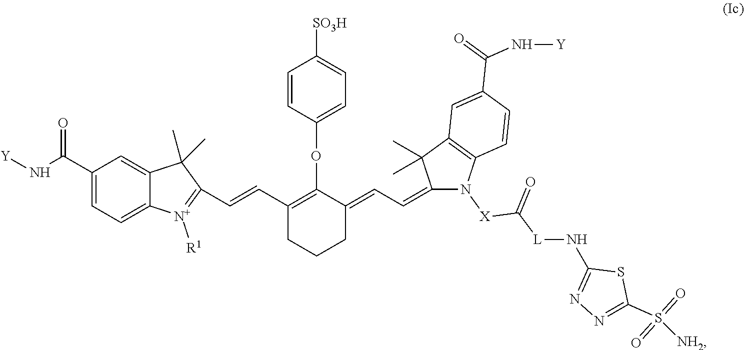
wherein R¹, X, Y an L are as defined in claim 1.

11. A method for the detection and demarcation of tumor tissue during diagnostic, interventional imaging and intraoperative procedures in a subject in need thereof comprising administering to the subject a compound of formula (I) according to claim 1, irradiating a body of the subject with a wavelength, and visualizing said compound.

12. The method according to claim 11 wherein the detection and demarcation of the tumor tissue is carried out under near-infrared (NIR) radiation.

13. The method according to claim 11, wherein said tumor is a tumor selected from brain cancer, breast cancer, head and neck cancer, ovarian cancer, prostate cancer, esophageal cancer, skin cancer, gastric cancer, pancreatic cancer, bladder cancer, oral cancer, lung cancer, renal cancer, uterine cancer, thyroid cancer, liver cancer, and colorectal cancer.

14. A pharmaceutical diagnostic composition comprising a compound of formula (I) as defined in claim 1 and at least one pharmaceutically acceptable carrier or excipient.

15. A diagnostic kit comprising at least one compound of formula (I) as defined in claim 1 together with additional adjuvants thereof for implementing a biomedical optical imaging application.

16. A diagnostic kit comprising a pharmaceutical composition as defined in claim 14 together with additional adjuvants thereof for implementing a biomedical optical imaging application.
